(19) United States
(12) Patent Application Publication
(10) Pub. No.: US 2007/0031342 A1
(43) Pub. Date: Feb. 8, 2007

(54) Sustained release microparticles for pulmonary delivery

(75) Inventors: Stelios T. Tzannis, Newark, CA (US); Negar Sadrzadeh, San Carlos, CA (US); Helen Schiavone, Freeland, WA (US); Renee Labiris, Paris (CA)

Correspondence Address: Michael Einschlag;NEKTAR THERAPEUTICS, 150 Industrial Road, San Carlos, CA, 94070, US

(73) Assignee: Nektar Therapeutics

(21) Appl. No.: 11/430,665

(22) Filed: May 9, 2006

Related U.S. Application Data
(60) Provisional application No. 60/651,489, filed on May 12, 2005.

(51) Int. Cl. A61L 9/04; A61K 38/23; A61K 31/573

(52) U.S. Cl. 424/45; 514/12; 514/171; 977/906

(57) ABSTRACT

A composition of microparticles for delivery to the pulmonary system provides sustained release of a pharmaceutical agent. The microparticles comprise a lipid structural matrix comprising a multilamellar structure of lipid bilayers having lipid chains ordered in an L_{βL} phase. The lipid matrix at least partially encapsulates the pharmaceutical agent at a bilayer interface formed between head groups of adjacent lipid layers. The microparticles are prepared by heating a precursor formulation comprising a solvent, matrix-forming excipient and pharmaceutical agent to a temperature above the liquid-crystalline transition temperature T_{c} of the matrix-forming excipient and below the melting or denaturation point of the pharmaceutical agent. The solvent is then removed to form microparticles with partially encapsulated pharmaceutical agent.

### CROSS-REFERENCE TO RELATED APPLICATIONS

The present application is a continuation of U.S. Provisional Application No. 60/651,489, filed on Jun. 22, 2005, which was converted from application Ser. No. 11/127,854, filed May 12, 2005; both of which are incorporated by reference herein and in their entireties.

### BACKGROUND

Embodiments of the invention relate to the pulmonary delivery of microparticles containing pharmaceutical agents and their methods of delivery and manufacture.

In inhalable drug delivery, aerosolized microparticles containing a pharmaceutical agent are orally or nasally inhaled to deliver the composition directly to a patient's respiratory tract or lungs. A pharmaceutical agent is any compound or composition capable of providing a beneficial or therapeutic effect on a patient. The pulmonary delivery microparticles are aspired though the peripheral airways to transport the pharmaceutical agent into a desired portion of the trachea or lung. Delivery by inhalation can provide rapid assimilation of a pharmaceutical agent owing to the high surface area and blood perfusion of the trachea and lungs. Typically, the pulmonary delivery microparticles have aerodynamic shapes and sizes that are tailored to allow transport to a desired pulmonary region.

Sustained release pulmonary delivery microparticles are being developed to provide sustained release of pharmaceutical agents in a pulmonary region to achieve a desirable optimal local or systemic drug levels and the appropriate pharmacologic response. Generally, sustained release provides a patient with continued exposure of the pharmaceutical agent in small dosages, without requiring the patient to take multiple daily doses which typically presents appreciable patient compliance risks. However, the preparation of sustained release microparticles is challenging, as it requires the retardation of dissolution and the control of the release kinetics of active ingredient from the small microparticles to be dispersed into the pulmonary regions. The combination of the large surface area provided by the small microparticles and the high levels of blood perfusion to the pulmonary organs typically results in rapid dissolution of the microparticles making sustained delivery of drugs using such microparticles difficult to attain. Furthermore, it is even more difficult to achieve sustained release while still maintaining the shape and size, and consequently the dispersibility and stability, of the microparticles.

Thus it is desirable to have microparticles comprising pharmaceutical agents that are readily aerosolizable and can provide adequate sustained release levels. It is further desirable to be able to deliver a pharmaceutical agent to a particular region of the pulmonary system without early or late entrapment in other pulmonary regions. It is also desirable for the pharmaceutical composition to be stable during storage, at especially at room temperatures.

### SUMMARY

A composition for pulmonary delivery includes microparticles comprising a pharmaceutical agent and a lipid matrix comprising a multilamellar structure of lipid bilayers having lipid chains ordered in an L_{βL} phase, the multilamellar structure at least partially encapsulating the pharmaceutical agent at a lipid bilayer interface formed between a plurality of head groups of adjacent lipid bilayers, and capable of providing sustained release dosage of the pharmaceutical agent. For example, the multilamellar structure can provide sustained release of the pharmaceutical agent at a rate of least about 1 mg/hr and for a time period of at least about 2 hours.

In one version, the lipid bilayers comprise phospholipid bilayers. The phospholipid layers can have parallel and tilted lipid chains ordered in the L_{βL} phase. In one version, the pharmaceutical agent is at least partially encapsulated in a linear interface gap formed between a first set of head groups of a first phospholipid layer and a second set of head groups of a second phospholipid layer, the first and second phospholipid layers being substantially parallel to one another about the linear interface gap.

In another version, the multilamellar structure at least partially encapsulates the pharmaceutical agent in an I-shaped interface gap between a first set of head groups of a first phospholipid layer and a second set of head groups of a second phospholipid layer, the first and second phospholipid layers curling in opposing directions about the I-shaped interface gap.

In yet another version, the multilamellar structure at least partially encapsulates the pharmaceutical agent between phospholipid bilayers comprising non-liposomal structures that are disposed in a lineal arrangement which is absent rotational symmetry.

Preparation of microparticles suitable for pulmonary delivery involves preparing a precursor formulation comprising at least one solvent, a matrix-forming excipient, and a pharmaceutical agent. The precursor formulation is heated to a temperature above the liquid-crystalline transition temperature T_{c} of the matrix-forming excipients and below the melting or denaturation point of the pharmaceutical agent. The solvent is then removed to form microparticles comprising a multilamellar structure of the matrix-forming excipients that at least partially encapsulates the pharmaceutical agent.

### DRAWINGS

These features, aspects and advantages of the present invention will become better understood with regard to the following description, appended claims, and accompanying drawings, which illustrate examples of the invention. However, it is to be understood that each of the features can be used in the invention in general, not merely in the context of the particular drawings, and the invention includes any combination of these features, where:

FIGS. 1A and 1B are schematic diagrams showing the L_{β′} multilamellar lipid phase with the lipid chains ordered and tilted; and the L_{α′} multilamellar lipid phase composed of melted lipid chains without tilt with respect to normal, respectively;

FIGS. 2A to 2C are top-views of chains in the plane of the membrane in which the dashed ellipses indicate the direction of tilt of the chains for the L_{βF} phase, the L_{βL} phase; and L_{βl} phase, respectively;

FIG. 3 is a graph showing X-ray diffraction patterns of the 2% w/w (solid line) and 5% w/w (dashed line) of microparticles comprising a DSPC structural matrix encapsulating budesonide;

FIGS. 4A and 4 B are schematic diagrams showing a multilamellar structure comprising the L_{β′} lipid phase encapsulating a pharmaceutical agent in an interface region between the heads of a bilayer of lineal lipid chains as shown in FIG. 4A, and in an interface region between the heads of a bilayer of curved lipid chains as shown in FIG. 4B.

FIG. 5 is a graph showing rabbit serum pharmacokinetics of sustained release microparticles comprising 20% w/w sCT in a matrix comprising a DPI composition (20 mg) as compared to 20% w/w sCT control (10 mg) following a single intratracheal aerosol administration;

FIGS. 6A to 6D show scanning electron micrographs of sustained released microparticles comprising 2% w/w budesonide encapsulated in a DSPC lipid matrix (6A and 6B) and a DPPC lipid matrix (6C and 6D);

FIG. 7 is graph of dissolution profiles of microparticles in a Survanta dissolution medium, the microparticles comprising (a) DPPC matrix with 2% w/w budesonide (∘), (b) DPPC matrix with 5% w/w budesonide (●), (c) DSPC matrix with 2% w/w budesonide (□), (d) DSPC with 5% w/w budesonide (▪) and (e) micronized budesonide control (Pulmicort powder) (Δ);

FIG. 8 is graph of dissolution profiles of microparticles comprising budesonide represented as the percent of drug remaining to be released as a function of the square root of time, as corrected for the immediate drug burst at t=0, where the lines represent linear fits to the data for the a) DPPC matrix with 2% w/w budesonide (∘), (b) DPPC matrix with 5% w/w budesonide (●), (c) DSPC matrix with 2% w/w budesonide (□) and (d) DSPC with 5% w/w budesonide (▪);

FIG. 9A is a graph of the plasma budesonide concentration versus time profile for microparticles comprising lipid matrix of DSPC or DPPC at different concentrations encapsulating budesonide versus Pulmicort powder (Astra Zeneca) in rats following intratracheal instillation; and

FIG. 9B is a graph of the mean (SD) cumulative amount of budesonide absorbed vs. time following intratracheal administration for microparticles comprising lipid matrix of DSPC or DPPC at different concentrations encapsulating budesonide versus Pulmicort powder.

### DESCRIPTION

Sustained release microparticles for pulmonary delivery provide sustained release dosing of a pharmaceutical agent to the pulmonary system at predictable dosage rates to achieve desirable local or systemic pharmaceutical agent levels and the resultant pharmacologic response. Although embodiments of the sustained release microparticles and their formulation are illustrated in the context of a dry powder composition of microparticles made from a liquid precursor formulation, the sustained release microparticles, precursor formulation, and delivery method, can be changed or used in other processes and systems, for example, non-pulmonary delivery or rapid dissolution systems; thus, the scope of the invention should not be limited to the illustrative examples provided herein.

The sustained release pulmonary delivery microparticles comprise a structural matrix composed of a matrix-forming excipient and a pharmaceutical agent that is at least partially encapsulated by the structural matrix. The structural matrix at least partially surrounds the pharmaceutical agent and provides a support structure having desirable aerodynamic and bulk density properties that allow pulmonary delivery. In one embodiment, the microparticles comprise a structural matrix composed of layers of lipid chains that form bilayer membranes which are adjacent to one another in the structure. The structural matrix at least partially encapsulates a pharmaceutical agent, which may be a single compound or a mixture of compounds that provides some therapeutic or beneficial effect on a patient. Exemplary lipids that can form the structural matrix and different pharmaceutical agents are described herein.

The structural matrix of lipid bilayers provides a rate of sustained release of the pharmaceutical agent from its surrounding matrix that is governed by (i) the physicochemical properties of the lipid structure, in particular the lipid chain length, transition temperature and lipid phase, (ii) the molecular geometry of the pharmaceutical agent, and (iii) the location of the pharmaceutical agent within the lipid matrix. Typically, the longer the lipid chain length and the higher the transition temperature of the lipid structure, the slower the dissolution or permeation rate of the encapsulated pharmaceutical agent in the lipid structural matrix. The dissolution rate of the pharmaceutical agent also decreases with increased length of the lipid chain, due to slower ‘diffusion’ of the agent out from a lipid matrix formed of longer chain lipids. Agent diffusivity also largely depends on the degree of disorder of the lipid bilayer, which determines its permeability. This process is expected to be largely dependent on the (T_{c}) in the composition. The lipid's transition temperature T_{c} of the bilayers in the matrix compositions also affects the rate of dissolution of the drug into the pulmonary organs.

The molecular structure and geometry of the pharmaceutical agent also affects their dissolution rate in the pulmonary regions, which in turn would affect sustained release dosage rates. The location of the pharmaceutical agent within the lipid matrix affects the degree to which the pharmaceutical agent is exposed to the external pulmonary surface, with a more encapsulated drug dissolving or permeating through the surrounding lipid structure at a slower rate than a drug composition located at or near the surface of the lipid structure. Thus, a number of different parameters can be adjusted to control the sustained release rates obtained from the pulmonary delivery microparticles.

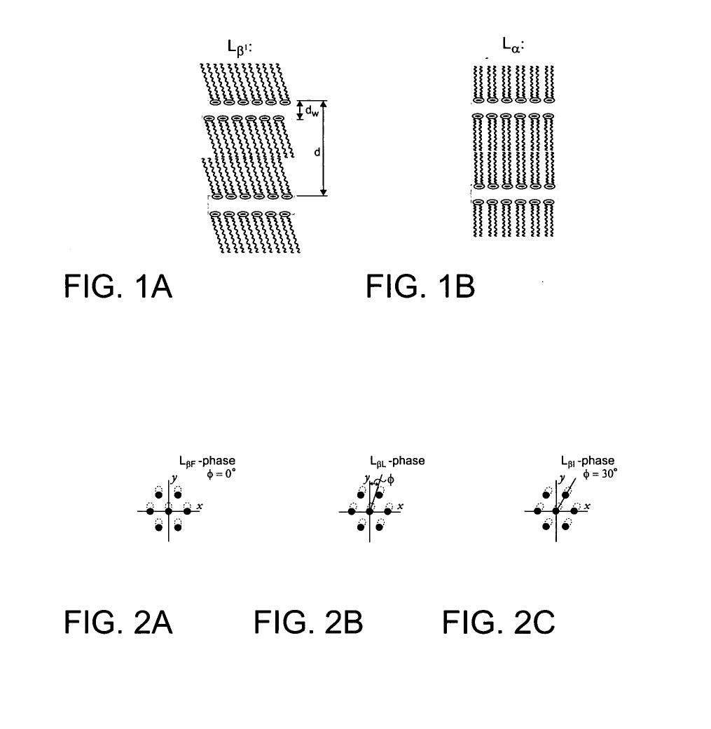

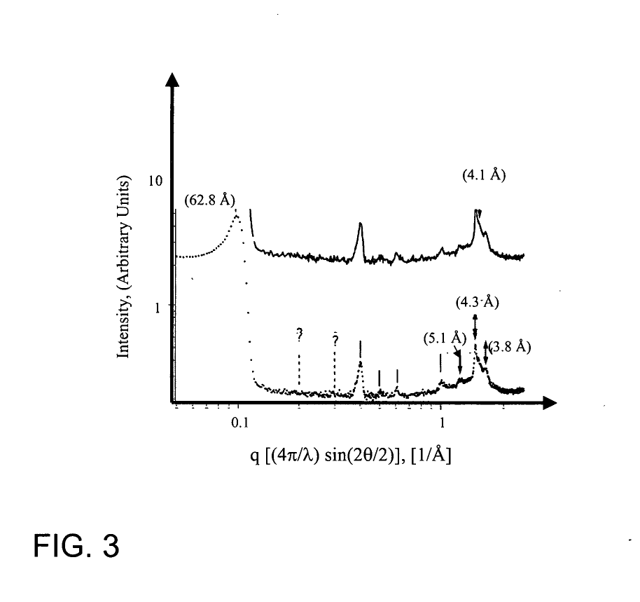

In one embodiment, sustained delivery microparticles having structural matrices that comprise a lipid membrane structure present in an L_{β′} phase, as for example, schematically illustrated in FIG. 1A, were found to provide good sustained release rates. In the L_{β′} phase, the lipid chains are ordered with tight lateral packing and tilted with respect to the lipid-bilayer normal. In contrast, the L_{α} phase as shown in FIG. 1B, comprises non-tilted, disordered lipid chains. Different lipids, such as phospholipids can be used to form this structure. The L_{β′} multilamellar phase of lipids comprises at least three distinct sub-phases depending on the direction of tilt of the ordered chains as, for example, illustrated in FIGS. 2A to 2C. The dashed ellipses indicate the direction of tilt of the chains. In the L_{βl} phase the chains are tilted towards their nearest neighbors, while in the L_{βF} phase the chains are tilted between the nearest neighbors (along the y-axis). In the L_{βL} phase the tilt direction of the chains is somewhere between that of the L_{βF} and L_{βl} phases. The L_{βL} phase lipid chains are tilted with respect to a normal to the lipid bilayer interface at a tilt angle of at least 15°, for example, at a tilt angle of about 30°. The laterally packed chains form a distorted rectangular phase with a lateral spacing between lipid chains of from about 3 Å to about 6 Å, and more specifically from 3.8 Å to 4.3 Å. Although the chains did not appear to form a 3-dimensional crystal, which is the most ordered and dry phase possible for the chains, they exist in the next most-ordered conformation possible for the lateral packing of the chains in the lipid bilayer.

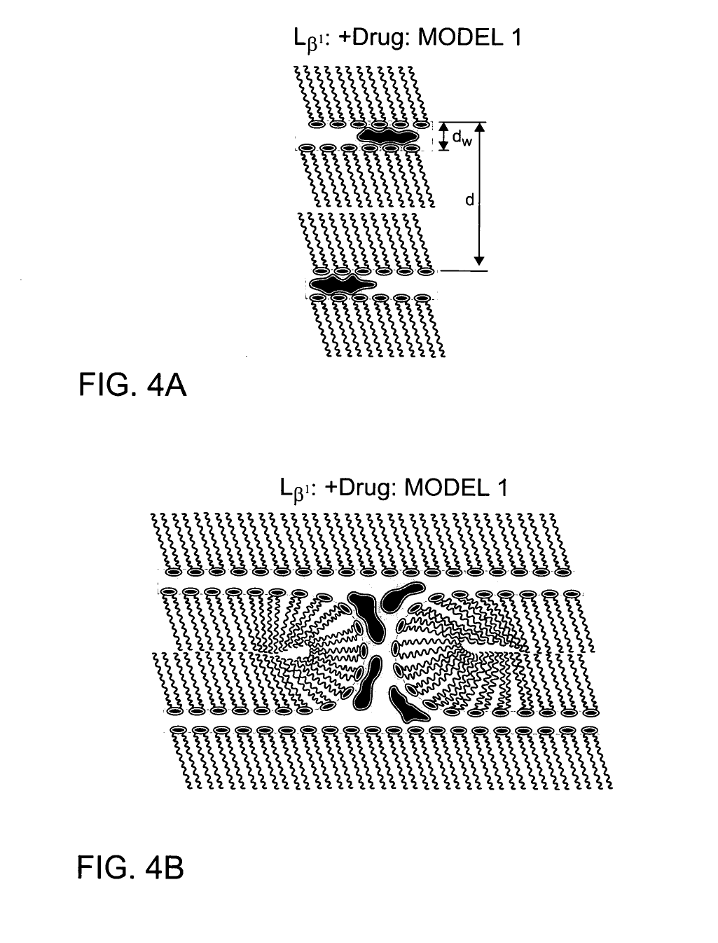

The X-ray diffraction data for the sustained release microparticles comprising lipid membrane structure present in an L_{β′} phase, revealed an unexpected, novel multilamellar lipid structure that at least partially encapsulates the pharmaceutical agent with lipid chains. Typically, the L_{βF} and L_{βl} phases both show two diffraction peaks in the high angle region whereas the L_{βL} phase shows three peaks, which are consistent with X-ray diffraction data. Exemplary small angle X-ray diffraction patterns for sustained release microparticles comprising two compositions of different phospholipid matrices encapsulating pharmaceutical agents are shown in FIG. 3. The X-ray intensities are plotted versus q=(4π/λ)sin(2θ/2) where λ=1.54 Å and 2θ is the scattering angle between the incident X-ray beam and the diffracted x-ray beam. In this diffraction pattern, the weak peak at a lattice spacing distance of 5.1 Å, as indicated with a single arrow, represents locally “melted chains” of curved regions of the lamellar lipid structure. The first peak at q₍₀₀₁₎=0.1 (1/Å) corresponds to the (001) peak of the (00L) series; other peaks of the (00L) series at q(004)=0.4 (1/Å), q(005)=0.5 (1/Å), q(006)=0.6 (1/Å), and q(0010)=1.0 (1/Å), are also indicated by solid lines in the figure. These peaks arise from the multilamellar structure of the lipids in both compositions with an inter-lamellar spacing of d=^{2π}/_{q(001)}=62.8 Å, as shown schematically in FIGS. 1A and 1B. The appearance of the same set of X-ray diffraction peaks in several microparticle compositions with different lipids implies that the same structures or common variants of thereof, can be expected for other sustained release multilamellar lipid structures.

The X-ray diffraction analysis also indicated that the pharmaceutical agent molecules are not inserted deep in the hydrophobic region of the chains, but instead are located between the lipid bilayers, because the lipid chains remain in the highly ordered L_{β′} phase. If the pharmaceutical agent were located deep in the hydrophobic region of the lipid bilayer chains, the sharp X-ray diffraction peaks at 4.3 Å, 4.1 Å, and 3.8 Å would be replaced by a broad peak characteristic of disordered lipid chains, since the location of the pharmaceutical agent would disrupt the periodic lattice structure of the lipid chains in the bilayers.

Furthermore, the presence of a broad and not narrow X-ray diffraction peak at approximately 5.1 Å is believed to result from the interaction of the pharmaceutical agent molecule with the surrounding lipid structure. However, the X-ray diffraction analysis of the lipid matrix structure does not support direct interaction of the pharmaceutical agent with the lipid bilayers of the microparticle; otherwise, the peaks would not be recognizable. Thus, it is believed that the pharmaceutical agent is clustered within small, inter-bilayer interface spaces having a dimension less than 3 Å, which corresponds to a highly dehydrated state that does not disturb the lattice ordering of the lateral lipid chains.

The model of the microparticles comprising a structural matrix of lipid bilayers with intercalated pharmaceutical agent is further supported by the Higuchi-type kinetics displayed by the lipid compositions in vitro, as described in the examples below. The diffusion-controlled release mechanism is typically provided by microparticles comprising at least partially encapsulated pharmaceutical agent. In the sustained release microparticles, entrapment of pharmaceutical agent increased with decreasing aliphatic chain length of the lipid matrix forming excipient. This effect presumably occurs due to decreased chain-chain interactions and the increased number of voids in microparticles having longer chain lengths.

Based on the above data, two possible structural models were constructed. While the proposed models illustrate exemplary versions of the lipid matrix structure and location of pharmaceutical agent in the matrix, they should not be used to limit the scope of the present invention, and other matrix structures or configurations of matrix structures and pharmaceutical agent, are encompassed in the scope of the present invention.

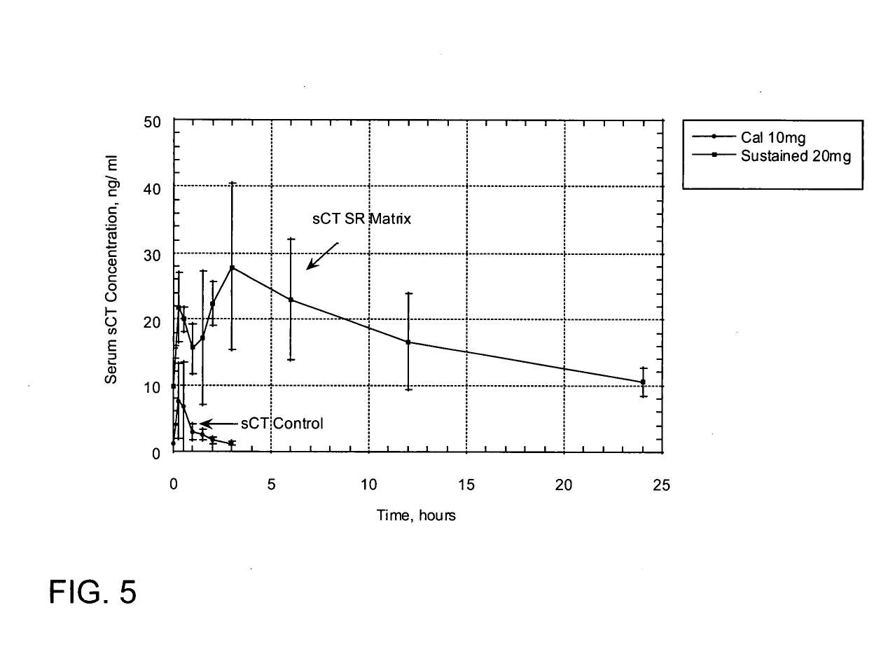

In structural model 1, it is assumed that the pharmaceutical agent has a narrow-shape with small dimensions which allows it to fit in the tight space in-between two layers of a phospholipid bilayer. It is believed that in this model, the lipid bilayer interface comprises a linear gap between adjacent lipid bilayers which are substantially parallel to one another. The lipid comprises a multilamellar structure with lipid bilayers which are in a lineal arrangement and substantially parallel to one another, as for example shown in FIG. 4A. For example, the bilayer interface can have a linear gap with a thickness of less than 3 Å, to accommodate a pharmaceutical agent having a dimension which is also less than 3 Å. An example of such a pharmaceutical agent is budesonide. Each bilayer comprises a first set of head groups of a first phospholipid layer and a second set of head groups of a second phospholipid layer. The bilayer forms a lineal arrangement with lipid chains that are substantially parallel to one another and separated by a linear interface gap. The phospholipid matrix at least partially encapsulates the pharmaceutical agent between the first head groups of the first phospholipid layer and the second head groups of the second phospholipid layer.

Model 2 assumes a significantly larger pharmaceutical agent molecule that does not easily fit in the linear gap of the interface between the adjacent bilayers. It is believed that in this model, the association of the pharmaceutical agent with the hydrophilic interface leads to the formation between adjacent phospholipid bilayers of an bilayer interface that forms an I-shaped gap in which at least a portion of the pharmaceutical agent is encapsulated as shown in FIG. 4B. Typically, the I-shaped gap has a thickness dimension of about greater than 3 Å, and consequently, can accommodate larger pharmaceutical agents having dimensions that are also larger than 3 Å. The I-shaped gap is formed between two opposing curved defect regions that occur due to the disruption of the lateral ordering of the chains in these local regions with the remainder of the chains in the flat regions of the membrane in the ordered L_{βL} state. These locally “melted chains” of the curved regions would give rise to a weak peak at a lattice spacing distance of 5.1 Å as observed in FIG. 3. In this phase, the multilamellar structure of the phospholipid matrix at least partially encapsulates the pharmaceutical agent between a first curved section comprising a first set of head groups of a first phospholipid layer, a second curved section comprising a second set of head groups of a second phospholipid layer, and the a linear section comprising a third set of head groups of a third phospholipid layer. The first and second curved sections of the phospholipid layers curl in opposing directions to define the I-shaped gap therebetween.

Thus, the observed structural matrices comprising multilamellar structures of lipid bilayers typically comprise non-liposomal structures that at least partially encapsulate pharmaceutical agent between the bilayers to prolong the duration of release of the pharmaceutical agent. Liposomal structures are generally circular with a rotational axis of symmetry, such as a sphere or annular structure. In contrast, the present non-liposomal structures typically have bilayers that are in a lineal or curvilinear arrangement. Adjacent lipid layers form a linear structure that has a large dimension in X-Y plane and a relatively smaller dimension in the Z-axis normal to the X-Y plane. The non-liposomal structures can even be substantially absent rotational symmetry. For example, the non-liposomal structures can have a linear shape in which both lipid layers of the bilayer structure are substantially parallel to one another, and form a sheet-like structure which may be in a flat plane, wavy, or curled up over itself. The sheet-like bilayer structure can have both linear sections and curved sections, such as a layer curled up over itself facing another layer curled in the opposite direction, and with overlying flat layers. The non-liposomal structures can also form an ellipsoid shaped structure, such as a compressed or flattened out sphere.

In the models and structures described herein, the pharmaceutical agent is at least partially encapsulated in the matrix structure formed by a matrix-forming excipient. For example, the pharmaceutical agent may be trapped between or within bilayers, trapped in an interaction with the polar head groups, or at least partially trapped and encapsulated by a combination of such mechanisms. The encapsulation of pharmaceutical agent within the matrix-forming excipient increases the amount of time the pharmaceutical agent is retained in the lungs, for example, by prolonging the dissolution of the pharmaceutical agent in vivo. The desired release time of the pharmaceutical agent depends on its type, dosage, activity, and on the type of matrix-forming excipients in the composition and their proportions. In addition, the relative proportions of matrix-forming excipient component and pharmaceutical agent can be adjusted so that a desirable amount of the pharmaceutical agent is encapsulated. This degree of encapsulation may also be such that the pharmaceutical agent may have some immediate activity and some sustained activity.

The efficiency of encapsulation of the pharmaceutical agent in the matrix-forming excipient can be increased by selecting suitable processing conditions that facilitate incorporation of the pharmaceutical agent in the matrix-forming excipient. In particular, process conditions that promote the liquid crystalline state of the matrix-forming excipient over the more highly ordered gel phase can substantially increase the encapsulation efficiency of pharmaceutical agent. The phase of the matrix-forming excipient is governed by a gel-to-crystalline transition temperature (T_{c}), also sometimes referred to as a melting temperature that is a characteristic property of the matrix forming excipient. For example, in the case of lipids such as phospholipids, the gel-like state at temperatures below T_{c} is characterized by close packing and increased van der Waals contacts between neighboring phospholipids, which reduces the mobility of the phospholipids. At T_{c}, a phase transition occurs that yields a more mobile and even more liquid-like crystalline phase. The phase transition is typically a sharp transition indicating a cooperative transition among the molecules. The “liquid crystal” phase at temperatures above T_{c} is characterized by more disordered phospholipids due to disruption of packing of the hydrocarbon tails of the phospholipids. With regards to encapsulation of the pharmaceutical agent, the high degree of order of the matrix-forming excipient in the gel state results in a high packing density of the excipient that does not favor infiltration of the pharmaceutical agent into the excipient structure. Accordingly, to facilitate encapsulate pharmaceutical agent into the matrix-forming excipient, process conditions are selected to promote less ordered liquid crystalline state of the matrix-forming excipient, which is more conducive to encapsulation of the pharmaceutical agent.

In one encapsulation method, processing conditions are selected to promote the liquid crystalline state of the matrix-forming excipient by maintaining a precursor formulation comprising the excipient at or above its gel-to-liquid crystal transition temperature T_{c}. The precursor formulation may be a liquid such as a solution, course suspension, slurry, paste, or colloidal dispersion such as an emulsion, reverse emulsion, microemulsion, multiple emulsion, particulate dispersion, or slurry. The selected temperature promotes the liquid-crystalline phase of the matrix-forming excipient in the precursor formulation, and thus, increases the ability of the matrix-forming excipient to encapsulate a pharmaceutical agent added to the solution. In one version, a solution comprising a mixture of the pharmaceutical agent and matrix-forming excipient is incubated for a pre-selected duration at a temperature that is at or above T_{c} to increase incorporation of the pharmaceutical agent in the matrix-forming excipient. In another version, a solution of the matrix-forming excipient may be heated at or above the temperature T_{c} before, or simultaneously with mixing of the pharmaceutical agent, to enhance the permeability and receptivity of the matrix-forming excipient for encapsulation of the pharmaceutical agent. However, the temperature of the combined solution is desirably maintained below the melting point or denaturation point of the pharmaceutical agent to inhibit decomposition or denaturing of the agent. This heating step is conducted before removal of the solvent from the solution. Other parameters of the solution containing the matrix-forming excipient can also be selected to promote the liquid-crystalline state of the matrix-forming excipient. For example, the solution may comprise a solvent that promotes the formation of the liquid-crystalline phase or can include additives that assist liquid-crystalline phase formation.

The solution comprising the pharmaceutical agent and matrix-forming excipient may further comprise a co-solvent system comprising first and second solvents combined in a volumetric ratio that provides better mixing of the pharmaceutical agent and matrix-forming excipient. For example, the composition and ratios of the first and second solvents may be selected to at least partially dissolve one or more components of the solution, such as for example, at least one of the pharmaceutical agent and matrix-forming excipient. At least one of the solvents may comprise a relatively less polar solvent that promotes the formation of more loosely organized matrix structures than would otherwise be formed in a solution comprising substantially only water, to promote the interdiffusion and mixing of the pharmaceutical agent with the more open and loosely structured matrix-forming excipient. In one version, the first solvent comprises a relatively polar solvent, such as for example, water. The second solvent can comprise a relatively less-polar solvent having a lower polarity than the first solvent, such as for example, at least one of an alcohol, a chlorinated solvent such as chloroform, ether or a fluorocarbon solvent. The first and second solvents are desirably at least partially miscible with one another. In a preferred version, the first solvent comprises water and the second solvent comprises ethanol.

Once the pharmaceutical agent has been mixed in the solution with the matrix-forming excipient, the liquid solution may be removed to provide dried particles comprising matrix-forming excipients having the pharmaceutical agent at least partially encapsulated therein. In one version, the solution comprises a co-solvent system that promotes the formation of encapsulating matrix structures during removal of the solution. For example, the liquid solution may comprise a co-solvent system having a first polar solvent in which the matrix-forming excipient is more soluble than the pharmaceutical agent, and a second solvent that is relatively less polar and that is removed more readily than the first solvent during the drying process. The higher solubility of the matrix-forming excipient in the less polar solvent results in the formation of organized matrix structures that at least partially surround the pharmaceutical agent as the less-polar solvent is removed from the solution to provide a solvent environment that is relatively more polar. The pharmaceutical agent is drawn into the matrix structures and away from the remaining solvent in which it is less soluble.

For example, for a solution comprising a first solvent comprising water and a second solvent comprising ethanol, the ethanol component will typically be removed more quickly during a drying process than the water component due to the lower boiling point of the ethanol component. Thus, for a solution comprising a relatively more hydrophobic pharmaceutical agent and a matrix-forming excipient such as a lipid that is relatively more soluble in water than the pharmaceutical agent, the removal of the ethanol leaves behind a water-rich environment that promotes the formation of organized matrix structures, such as bilayer and vesicle structures, while the pharmaceutical agent associates with the more non-polar ends of the matrix-forming excipient and is drawn into the matrix structures and away from the water. Accordingly, the formation of matrix structures that at least partially encapsulates the pharmaceutical agent is facilitated by providing the proper solvent environment during the drying process. A further description of such co-solvent systems and their use in promoting pharmaceutical agent encapsulation is provided in PCT Publication No. PCT/US04/16696, entitled “Pharmaceutical Formulation Comprising a Water-insoluble Active Agent,” filed on May 27, 2004, which is herein incorporated by reference in its entirety.

In one method of preparing the precursor formulation, a first solution is formed by at least partially dissolving the matrix-forming excipient in a first solvent, such as ethanol, which is heated to a temperature at or above the T_{c} of the matrix-forming excipient. A second solution is also formed by at least partially dissolving a pharmaceutical agent in a second solvent, such as buffered water. For example, the second solvent can comprise water having at least one of phosphate, Tris-HCl, citrate, borate and caodylate buffers. The second solution may also include drug-solubilizing excipients that facilitate dissolution of the pharmaceutical agent in the second solution and may also comprise a glass-forming excipient. The second solution may be heated to a temperature that is at or above the T_{c} of the matrix-forming excipient but also preferably below the melting point or denaturation point of the pharmaceutical agent.

The first and second solutions are combined to form a third solution at volumetric ratios selected to provide optimal mixing of the pharmaceutical agent and matrix-forming excipient. A suitable volumetric ratio of the first solution (ethanol) to the second solution (water) may be a ratio of from about 99.9:0.1 to about 1:100, such as from about 70:30 to about 30:70, and even from about 50:50 to about 70:30. The temperature of the solution comprising the mixture of the matrix-forming excipient and pharmaceutical agent is desirably maintained at a pre-selected mixing temperature that is above the liquid-crystalline transition temperature T_{c} of the matrix-forming excipient, while still remaining below the melting or denaturation point of the pharmaceutical agent. The liquid-crystalline phase of the matrix-forming excipient is thus facilitated in the solution, and the pharmaceutical agent can more readily associate and mix with the more accessible and open liquid-crystalline phase structure to promote encapsulation of the pharmaceutical agent. The solution comprising the mixture of the matrix-forming excipient and the pharmaceutical agent can be incubated at temperatures above the Tc and below the melting or denaturation point for a desired period of time to ensure optimum mixing and association of the matrix-forming excipient and pharmaceutical agent, for example, for at least about an hour and desirably about two hours. In the feed stock preparation step, the selected pharmaceutical agent is dissolved in a solvent, such as water, to produce a concentrated solution. Additives, such as the polyvalent cation may be added to the solution or may be added to the phospholipid emulsion as discussed below. The pharmaceutical agent may also be dispersed directly in the emulsion, particularly in the case of water insoluble agents. Alternatively, the drug may be incorporated in the form of a solid particulate dispersion. The concentration of the pharmaceutical agent used is dependent on the amount of agent required in the final powder and the performance of the delivery device employed.

In another encapsulation method, the pharmaceutical agent is suspended in a solution, and the matrix-forming excipient is mixed into the solution so that the particles of pharmaceutical agent are coated with the matrix-forming excipient. In this version, the first solution is formed by at least partially dissolving the matrix-forming excipient in a first solvent in which the pharmaceutical agent is substantially insoluble, such as for example ethanol, and the first solution is heated to a temperature at or above the T_{c} of the matrix-forming excipient. The pharmaceutical agent is suspended in a second solution comprising a second solvent, such as for example water, and the second solution is also heated to a temperature at or above the T_{c} of the matrix-forming excipient but preferably below the melting point or denaturation point of the pharmaceutical agent. The second solution may also comprise a glass-forming excipient. The first and second solutions are combined to form a third solution at a volumetric ratio selected to provide optimal mixing and coating of the pharmaceutical agent by the matrix-forming excipient, such as for example, a volumetric ratio of the first solution (ethanol) to the second solution (water) of from about 100:0.1 to about 20:80, and preferably from about 70:30 to about 30:70. In one version, the pharmaceutical agent may even be suspended in the same solution as the matrix-forming excipient, such as for example, in a solution of ethanol, substantially without providing a second solvent. The solution comprising the matrix-forming excipient and pharmaceutical agent may be incubated at one or more selected temperatures above the T_{c} and below the melting or denaturation point for a desired period of time to ensure optimum mixing and association of the matrix-forming excipient and pharmaceutical agent.

In another method the liquid precursor formulation contains a matrix-forming excipient that is a phospholipid modified by a polyvalent cation. In this version, the pharmaceutical agent is dissolvent in a suitable solvent such as water. A second liquid composition comprising an oil-in-water emulsion containing a polyvalent cation is formed in a separate vessel. The oil employed is preferably a fluorocarbon (e.g., perfluorooctyl bromide, perfluorooctyl ethane, perfluorodecalin) which is emulsified with a phospholipid. For example, polyvalent cation and phospholipid may be homogenized in hot distilled water (e.g., 60° C.) using a suitable high shear mechanical mixer (e.g., Ultra-Turrax model T-25 mixer) at 8000 rpm for 2 to 5 minutes. Typically 5 to 25 g of fluorocarbon is added dropwise to the dispersed surfactant solution while mixing. The resulting polyvalent cation containing perfluorocarbon in water emulsion is processed using a high pressure homogenizer to reduce the particle size. Typically the emulsion is processed at 12,000 to 18,000 psi using 5 discrete passes and kept at 50 to 80° C. The pharmaceutical agent solution and perfluorocarbon emulsion are then combined to form a precursor solution, or fed separately and directly into a drying system. Typically the two preparations will be miscible as the emulsion will preferably comprise an aqueous continuous phase. While the pharmaceutical agent is solubilized separately in this example, the agent can also be solubilized or dispersed directly in the emulsion. In such cases, the active emulsion is dried without combining a separate agent preparation.

The precursor formulation comprising the matrix-forming excipient and the pharmaceutical agent in solution or suspension is then dried to remove the solvent form the solution. Drying may be conducted, for example, in a spray-drying process. The spray drying process results in microparticles that typically have a relatively thin porous wall defining a large internal void, however, other solid or porous structures can also be formed. The spray drying process is advantageous over other processes because the resultant microparticles are less likely to rupture during processing or de-agglomeration. The spray drying promotes the formation of matrix-forming structures that encapsulate the pharmaceutical agent by removing the less polar solvent, in this case ethanol, more rapidly than the water solvent, providing water-rich droplets in which the matrix-forming excipient is more soluble than the pharmaceutical agent, thus, forming matrix structures such as bilayers and vesicles that at least partially surround and insulate the pharmaceutical agents from the water solutions.

The drying process converts the liquid precursor formulation to a dry powder comprising microparticles. To remove the solvent, the precursor feedstock is heated to a temperature of at least the evaporation temperature of the solvent. In one process, the liquid composition is dispersed into a sufficient volume of hot gas, such as air, to evaporate and dry the liquid droplets. Typically, the feedstock is sprayed into a current of warm filtered air that evaporates the solvent and conveys the dried product to a collector. The spent air is then exhausted with the solvent. In one version, the spray drying process is conducted using warm dry air maintained at a temperature that is within a range between the T_{c} of the matrix-forming excipient and the melting or denaturation point of the pharmaceutical agent. Operating conditions such as inlet and outlet temperature, feed rate, atomization pressure, flow rate of the drying air, and nozzle configuration can be set to produce the required particle size, and production yield of the resulting dry particles. Exemplary settings are as follows: an air inlet temperature between 60 and 170° C.; an air outlet between 40° C. and 120° C.; a feed rate between 3 and 15 ml/min; an aspiration air flow of 300 l/min; and an atomization air flow rate between 25 to 50 l/min. Commercial spray dryers manufactured by Buchi Ltd. or Niro Corp. can be used to produce the pharmaceutical composition. Examples of spray drying methods and systems suitable for making the dry powders of the present invention are disclosed in U.S. Pat. Nos. 6,077,543, 6,051,256, 6,001,336, 5,985,248, and 5,976,574, all of which are incorporated herein by reference in their entireties.

The dispersion stability and dispersibility of the spray dried particulate compositions can be improved using a blowing agent, as described in WO 99/16419 cited above. This process forms an emulsion, optionally stabilized by an incorporated surfactant, typically comprising submicron droplets of water immiscible blowing agent dispersed in an aqueous continuous phase. The blowing agent may be a fluorinated compound (e.g. perfluorohexane, perfluorooctyl bromide, perfluorooctyl ethane, perfluorodecalin, perfluorobutyl ethane) which vaporizes during the spray-drying process, leaving behind generally hollow, porous aerodynamically light microspheres. Other suitable liquid blowing agents include nonfluorinated oils, chloroform, Freons, ethyl acetate, alcohols, hydrocarbons, nitrogen, and carbon dioxide gases. Although the particulate compositions are preferably formed using a blowing agent, the drying process can also be performed without adding blowing agent by spray drying an aqueous dispersion of the precursor formulation without adding blowing agents. In such cases, the pharmaceutical composition may possess special physicochemical properties, such as high crystallinity, elevated melting temperatures, surface activity, etc., that makes it particularly suitable for such techniques.

The precursor formulation can also be dried to form microparticles by a lyophilization process, which is a freeze-drying process in which water is sublimed from the composition after it is frozen. The advantage of this process is that biologicals and pharmaceuticals that are relatively unstable in an aqueous solution can be dried without elevated temperatures, and then stored in a dry state in which there are fewer stability problems. This technique is particularly compatible with the incorporation of peptides, proteins, genetic material and other natural and synthetic macromolecules in compositions without compromising physiological activity. The lyophilized cake containing a fine foam-like structure can be micronized using known techniques to provide the desired sized microparticles

The drying process results in dry powders composed of microparticles having a particle size selected to permit aerodynamic penetration into the trachea or alveoli of the lungs. For such delivery, a suitable mass median aerodynamic diameter of the microparticles is less than 5 μm, and preferably less than 3 μm, and most preferably from about 1 μm to about 3 μm. The mass median diameter of the microparticles may be less than 20 μm, more preferably less than 10 μm, more preferably less than 6 μm, and most preferably from about 2 μm to about 4 μm. The delivered dose efficiency (DDE) of these powders may be greater than 30%, and more preferably greater than 60%. The dry powders have a moisture content of less than 15% by weight, and more preferably less than 10% or even less than 5% by weight. Such particle sizes are described in WO 95/24183, WO 96/32149, WO 99/16419, WO 99/16420, and WO 99/16422, all of which are all incorporated herein by reference in their entireties. Mass median diameter (MMD) is a measure of mean particle size, since the microparticles are generally polydisperse (i.e., consist of a range of particle sizes). MMD values as reported herein are determined by centrifugal sedimentation and/or by laser diffraction, although any number of commonly employed techniques can be used for measuring mean particle size. Mass median aerodynamic diameter (MMAD) is a measure of the aerodynamic size of a dispersed particle. The aerodynamic diameter is used to describe an aerosolized microparticle powder in terms of its settling behavior, and is the diameter of a unit density sphere having the same settling velocity, generally in air, as the particle. The aerodynamic diameter encompasses particle shape, density and physical size of a microparticle. As used herein, MMAD refers to the midpoint or median of the aerodynamic particle size distribution of an aerosolized powder determined by cascade impaction.

The microparticles can also be hollow and/or porous microstructures, as described in the aforementioned in WO 99/16419, WO 99/16420, WO 99/16422, WO 01/85136 and WO 01/85137. The hollow and/or porous microstructures are particularly useful in delivering the pharmaceutical agent to the lungs because the density, size, and aerodynamic qualities of hollow and/or porous microparticles allow the particles to be transported into the trachea or deep lungs by inhalation. In addition, the hollow or porous microstructures reduce the attraction forces between particles, making the microparticles easier to deagglomerate during aerosolization and improving their flow properties. The hollow and/or porous microstructures may exhibit, define or comprise voids, pores, defects, hollows, spaces, interstitial spaces, apertures, perforations or holes, and may be spherical, collapsed, deformed or fractured particulates.

In one version, microparticles have a bulk density less than 0.5 g/cm³, more preferably less than 0.3 g/cm³, and sometimes less 0.1 g/cm³. By providing a low bulk density, the minimum powder mass that can be filled into a unit dose container is reduced, which eliminates the need for carrier particles. That is, the relatively low density of the microparticles provides for the reproducible administration of relatively low dose pharmaceutical compounds. Moreover, the elimination of carrier particles will potentially minimize throat deposition and any “gag” effect, since the large lactose particles will impact the throat and upper airways due to their size.

In some instances, it is desirable to deliver high dose, such as doses greater than 10 mg of pharmaceutical agent to the lung in a single inhalation. The described microparticles allow for doses greater than 10 mg, sometimes greater than 25 mg, to be delivered in a single inhalation. To achieve this, the bulk density of the powder is preferably less than 0.4 g/cm³, and more preferably less than 0.2 g/cm³. Generally, a drug loading of more than 5% w/w, more preferably more than 10% w/w, more preferably more than 20% w/w, more preferably more than 30% w/w, and most preferably more than 40% w/w is also desirable when the required lung dose in more than 10 mg.

The encapsulation of the pharmaceutical agent within the lipid matrix of the microparticles increases the time the agent is retained in the trachea or lungs. This is due to encapsulation of the agent in such a manner that extends release time life of the agent in the lungs compared to the agent's release time when it is not encapsulated. In one version, the microparticles comprise encapsulated pharmaceutical agent that is released over a time period of at least about 1 or 2 hours, in some cases at least about 3 or at least about 6 hours, and in other cases at least about 12 hours or at least about 24 hours. The desired sustained release time of the pharmaceutical agent depends on the agent, the dosage of the agent in the microparticle, the desired activity of the agent, and the types of lipids used in the composition and their proportions. For example, the microparticle with the encapsulated pharmaceutical agent can provide a sustained release dosage of the pharmaceutical agent of at least about 1 mg/hr for at least about 2 hours. In addition, the relative proportions of lipid and pharmaceutical agent can be adjusted in order for a desirable amount of the agent to become encapsulated.

The microparticles are delivered to the pulmonary air passages using aerosolization devices that aerosolize dry powders, propel liquid or powder with a propellant, or use a compressed gas to aerosolize a liquid or suspension. Dry powder inhalers (DPI) comprise dry powders that are inspired by the patient into respiratory tract and lungs. Metered dose inhalers (MDI) deliver medicaments in a solubilized or dispersed form using Freon or other relatively high vapor pressure propellant that forces aerosolized medication into the respiratory tract. Nebulizers deliver medicated liquids by forming an inhalable aerosol. A liquid dose instillation device delivers a liquid composition drop by drop into the pulmonary system. More recently, direct pulmonary delivery of drugs during liquid ventilation or pulmonary lavage using a fluorochemical has also been explored. Exemplary nebulizers are described in WO 99/16420, metered dose inhalers are described in WO 99/16422, liquid dose instillation apparatus are described in WO 99/16421, and dry powder inhalers are described in U.S. patent application Ser. No. 09/888,311 filed on Jun. 22, 2001, in WO 02/83220, and in U.S. Pat. No. 6,546,929 all of these patents and patent applications being incorporated herein by reference in their entireties.

The microparticles may be contained in a capsule that may be inserted into an aerosolization device. The capsule may be of a suitable shape, size, and material to contain the microparticles and to provide them in a usable condition. For example, the capsule may comprise walls made from a material that does not adversely react with the pharmaceutical composition of the microparticle. In addition, the wall may comprise a material that allows the capsule to be opened to allow its contents to be aerosolized. In one version, the capsule walls comprise one or more of gelatin, hydroxypropyl methylcellulose (HPMC), polyethyleneglycol-compounded HPMC, hydroxyproplycellulose, agar, or the like. The capsule can also have telescopically adjoining sections, as described for example in U.S. Pat. No. 4,247,066 which is incorporated herein by reference in its entirety. The size of the capsule may be selected to adequately contain the dose of the pharmaceutical composition. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Suitable capsules are available commercially from, for example, Shionogi Qualicaps Co. in Nara, Japan and Capsugel in Greenwood, S.C. After filling with microparticles, a top portion may be placed over the bottom portion to form the capsule shape and to contain the powder within the capsule, as described in U.S. Pat. Nos. 4,846,876, 6,357,490, and in the PCT application WO 00/07572 published on Feb. 17, 2000, all of which are incorporated herein by reference in their entireties.

In the microparticle compositions described herein, the pharmaceutical agent includes any agent, drug compound, composition of matter or mixture thereof, which provides some beneficial effect to a patient, including pharmacologic, therapeutic or other benefit; for example, foods, food supplements, nutrients, drugs, vaccines, vitamins, and other beneficial agents. The agent can also be a physiologically or pharmacologically active substance, or mixtures thereof, that produce a localized or systemic effect in a patient. The pharmaceutical agent can also be an inorganic or an organic compound, including without limitation, drugs which act on peripheral nerves, adrenergic receptors, cholinergic receptors, skeletal muscles, cardiovascular system, smooth muscles, blood circulatory system, synoptic sites, neuroeffector junctional sites, endocrine and hormone systems, immunological system, reproductive system, skeletal system, autacoid systems, alimentary and excretory systems, histamine system, and the central nervous system.

Suitable pharmaceutical agents may be selected from, for example, hypnotics and sedatives, psychic energizers, tranquilizers, respiratory drugs, anticonvulsants, muscle relaxants, antiparkinson agents (dopamine antagonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The pharmaceutical agent, when administered by inhalation, may act locally or systemically.

The pharmaceutical agent may also fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like. Examples of pharmaceutical agents suitable for use in this invention include but are not limited to one or more of calcitonin, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicillinase-sensitive agents like penicillin G, penicillin V, penicillinase-resistant agents like methicillin, oxacillin, cloxacillin, dicloxacillin, floxacillin, nafcillin; gram negative microorganism pharmaceutical agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Pharmaceutical agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, a pharmaceutical agent may comprise live attenuated or killed viruses suitable for use as vaccines. Other useful drugs include those listed within the Physician's Desk Reference (PDR 58^{th} Edition, 2004); which is incorporated herein by reference in its entirety.

Further examples of suitable pharmaceutical agents particularly suitable for sustained release include both locally-acting therapeutics, such as bronchodilators, anti-inflammatory agents, and corticosteroids; and also systemically delivered drug molecules, such as proteins, peptides and small molecules. Sustained release may also be desirable for pharmaceutical agents such as insulin, fluticasone propionate, testosterone, prostacycline, budesonide and antibiotics.

Sustained release of pharmaceutical agents that induce significant side effects in large doses would also be beneficial. For example, inhaled therapeutics which induce significant side effects include most bronchodilator β₂-agonists which often exert cardiovascular side effects, such as hypotension and tachycardia due to the stimulation of β₂-adrenoreceptors in the systemic circulation and cross-reactivity with cardiac β₂-adrenoreceptors. Sustained release of these pharmaceutical agents would offer a significant advantage in the treatment of chronic lung diseases, such as asthma, by prolonging drug retention in the targeted receptors, minimizing bio-distribution throughout the systemic circulation and thereby reducing the associated side effects. Sustained release dosage can also be used to deliver liposome-encapsulated amphotericin B (AmBiosome®) to reduce the high renal toxicity associated with its administration. Similarly, pulmonary delivery of other liposomal compositions can provide reduction of drug-related side effects attributed to the low systemic exposure by the prolonged local retention of the drug. Sustained release is also desired to regulate the drug absorption kinetics to maintain consistent drug levels in systemic circulation over time, as with basal insulin. Sustained release also allows reduction of the drug dose owing to the decrease of the systemic drug distribution.

In one version, the pharmaceutical agent comprises a molecule that exhibits at least amphiphilic properties, and which may even have a hydrophobic character, such as for example, peptides and small proteins, steroids and hydrophobic antibiotics. It may also include water insoluble agents that are optionally also crystalline and maintain their crystalline structure during processing into the microparticles. Examples of preferred pharmaceutical agents include, for example, salmon calcitonin (sCT) and budesonide. Salmon calcitonin positively influences bone mass density due to its inhibiting effect on osteoclast activity, and consequently, is used for treatment of osteoporosis, Paget's disease, hypercalcemia and reflex sympathetic dystrophy. Budesonide is a potent anti-inflammatory synthetic corticosteroid that is used to prevent wheezing, shortness of breath, and troubled breathing caused by severe asthma and other lung diseases and also management of nasal symptoms of seasonal or perennial allergic rhinitis in adults and children six years of age and older. Fungicides can also be included.

Pulmonary delivery of corticosteroids could be significantly enhanced by prolongation of their local lung action, reduced Cₘₐₓ-related side effects, possibly reduce dose and significantly improve patient convenience by reducing multiple daily dosings. An example of an anti-inflammatory corticosteroid that would benefit from prolonged retention in the lung is budesonide, which exhibits potent glucocorticoid and weak mineralocorticoid activity, and is used in the maintenance treatment of asthma in adult and pediatric patients. Currently, budesonide is administered twice daily as a micronized powder via a multi-dose dry powder inhaler (Pulmicort Turbuhaler, Astra USA), which exhibits fast absorption following inhalation delivery (tmax˜30 min). Inhaled budesonide is absorbed systemically, exhibiting an absolute bioavailability of 39%, which is higher than the nasal and oral bioavailabilities of 21±6% and 10.7±4.3%, respectively. However, its rapid absorption may also lead to short duration of clinical effects and possible removal of the active material via macrophages or mucociliary clearance, as well as a need for frequent dosing when low doses are used.

The amount of pharmaceutical agent provided by the sustained release microparticles is that amount needed to deliver a therapeutically effective amount of the pharmaceutical agent per unit dose to achieve the desired result. In practice, this will vary widely depending upon the particular agent, its activity, the severity of the condition to be treated, the patient population, dosing requirements, and the desired therapeutic effect. The microparticles can encapsulate anywhere from about 1% by weight to about 99% by weight pharmaceutical agent, typically from about 2% to about 95% by weight pharmaceutical agent, and more typically from about 5% to 85% by weight pharmaceutical agent, and will also depend upon the relative amounts of additives contained in the microparticles. The compositions of the invention are particularly useful for pharmaceutical agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one pharmaceutical agent may be incorporated into the compositions described herein and that the use of the term “agent” in no way excludes the use of two or more such agents.

The structure of the microparticle is formed by a matrix-forming excipient that is capable of associating with itself or other matrix-forming excipients to provide an at least partially ordered structure that encapsulates the pharmaceutical agent. Typically, the matrix-forming excipient comprises at least about 50%, and even about 70%, 80% or 90% by weight of the microparticle. In one example, when a matrix-forming excipient comprising a lipid is placed in a precursor formulation comprising a relatively polar solvent, the more non-polar ends of the lipids, such as more hydrophobic carbon chain tails, are oriented towards one another and away from the polar solvent; whereas, the more polar ends, for example ionic head groups, are oriented towards the solvent and may also be oriented away from one another, according to the type of matrix structure being formed. Upon drying, the resulting matrix structure may comprise for example, bilayers, micelles, Ivesicles, or combinations of these and/or other matrix structures formed by the organization and association of the matrix-forming excipient in response to hydrophobic/hydrophilic interactions with the solvent.

In one version, the matrix-forming excipient comprises at least one of a phospholipid, phosphoglycolipid, pegylated phospholipid, sterol, long-chain triglyceride, fatty acid and polymer. The saturated phospholipids may include, for example, saturated phospholipids having an acyl chain length of C14:0, C13:0, C12:0, C11:0, and C10:0 of phosphatidylcholine, phosphatyidyl ethanolamine, phosphatidylserine, phosphatidylglycerol, phosphatic acid, cholesterol and cardiolipin, and unsaturated phospholipids, such as dioleylphosphatidylcholine and natural unsaturated phospholipids, such as egg PC, and other phospholipids known in the art. Further examples include diarachidoylphosphatidylcholine dibehenoylphosphatidylcholine, diphosphatidylglycerol, short-chain phosphatidylcholines, long-chain saturated phosphatidylethanolamines, long-chain saturated phosphatidylserines, long-chain saturated phosphatidylglycerols, and long-chain saturated phosphatidylinositols. The phospholipid component serves as both the matrix for transporting the pharmaceutical agent and also as the source of molecules for encapsulation of the agent. Examples of phospholipid matrices are described in WO 99/16419, WO 99/16420, WO 99/16422, WO 01/85136 and WO 01/85137 and in U.S. Pat. Nos. 5,874,064; 5,985,309; and 6,503,480, all of which are incorporated herein by reference in their entireties.

In one preferred version, the matrix-forming excipient comprises a phosphilipid that has good biocompatibility, for example a saturated phospholatidylcholine (PC), such as dimyristoyl phosphatodylcholine (DMPC), dipalmitoyl phosphatidylcholine (DPPC), distearoyl phosphatidylcholine (DSPC), and diarachidonyl phosphatodylcholine (DAPC). Desirable phospholipids may also include saturated symmetric 1,2 dialkyl phospholipids such as 22:0 PC 1,2 dibehenoyl-phosphatidylcholine and other C16:0-C24:0 PC. Other desirable phospholipids may include saturated asymmetric 1, alkyl-2, alkyl phospholipids such as 1-palmitoyl, 2-stearoyl phosphatidylcholine, 1-stearoyl, 2-arachidonyl phosphatidylcholine and other C16-C24 lipid chain combinations. Further examples may include 1 palmitoyl-2, stearoyl phosphatidylcholine (with C16 and C18 chains) and 1 palmitoyl-2, eicosanoyl phosphatidylcholine. More preferably, the phospholipid, or mixture thereof, has a melting temperature of at least 32° C. These phospholipids are saturated, but unsaturated versions of these phospholipid types, or combinations of saturated and unsaturated types can also be used.

The matrix-forming excipient can also include a mixture of phospholipids selected to provide desirable transition temperature characteristics. For example, in one version dimyristoylphosphatidylcholine (DMPC) is combined with dipalmitoylphosphatidylcholine (DPPC) which has a hydrated liquid transition temperature of 42° C. In another version, one or more of the above listed phospholipids having a hydrated liquid transition temperature below 37° C. is mixed with one or more of the following phospholipids having a hydrated liquid transition temperature above 37° C., such as one or more of saturated phospholipids having an acyl chain length of C15:0, C16:0, C17:0, C18:0, C19:0, C20:0, C21:0, C22:0, C23:0, and C24:0 of phosphatidylcholine, phosphatyidyl ethanolamine, phosphatidylserine, phosphatidylglycerol, phosphatic acid, cardiolipin, and sphingolipids.

In yet another version, the lipid component of the matrix-forming excipient can include phospholipids combined with other non-phospholipid lipids, such as sterols, fatty acids, and their salts. Examples of sterols include cholesterol, ergosterol, and the like. Examples of fatty acids include saturated and unsaturated lipids of chain length C12 to C20, such as myristic, palmitic, stearic, eicosanoic, acid and salts thereof. Inclusion of cholesterol in the lipid component will stabilize the phospholipids bilayers by inserting itself between neighboring lipid chains, and thereby modifying the release of the entrapped active from the liposomal composition. Non-phospholipid vesicles can also be formed, for example, by mixtures of acid salts of quanternary amines, fatty alcohols and acids, fatty acid diethanolamines, ethoxylated fatty alcohols and acids, glycol esters of fatty acids, fatty acyl sarcocinates, glycerol fatty acid mono and diesters, ethoxylated glycerol fatty acid esters, glyceryl ethers and dimethyl amides.

Charged phospholipids may also be used, such as for example, the lipid component of the matrix-forming excipient may comprise one of more of phosphatidylglycerols, phoshatidylserine, phosphatidylinositols, and PEGylated derivatives thereof. Electrostatic repulsion between charged headgroups can increases interbilayer thickness, facilitating increases in solubilization capacity of the vesicular structures, thereby enabling higher drug loading and potentially increasing the encapsulation efficiency. The use of charged phospholipids may in some cases also facilitate increases in encapsulation and retention for oppositely charged pharmaceutical agents.

The matrix forming phospholipid content of the microparticles may be from 0.1% to 99.9%, preferably from 20% to 99%. The precise percentages are dependent on the pharmaceutical agent, the dose, the form of delivery, the desired degree of spontaneous encapsulation, and other factors. The pharmaceutical agent load accordingly. In one version, the phospholipid itself may be the pharmaceutical agent, such as when delivering natural or synthetic lung surfactant to the lungs.

Other matrix-forming excipients that can be used in combination with the phospholipid include, for example, surfactants, saturated and unsaturated lipids, long-chain triglycerides, fatty acids, non-ionic detergents and nonionic block copolymers. The surfactant may comprise fluorinated and non-fluorinated compounds. Nonionic detergents suitable as co-surfactants, include sorbitan esters including sorbitan trioleate, sorbitan sesquioleate, sorbitan monooleate, sorbitan monolaurate, polyoxyethylene, sorbitan monolaurate, and polyoxyethylene, sorbitan monooleate, oleyl polyoxyethylene ether, stearyl polyoxyethylene ether, lauryl polyoxyethylene ether, glycerol esters, and sucrose esters. Block copolymers include diblock and triblock copolymers of polyoxyethylene and polyoxypropylene, including poloxamer 188, poloxamer 407, and poloxamer 338. Ionic surfactants such as sodium sulfosuccinate, and fatty acid soaps may also be utilized. Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used when desirable.

A biocompatible copolymer or blend can also be used to improve the sustained delivery efficiency of the matrix-forming excipient of the microparticle structure and the stability of their dispersions. Potentially useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.).

By adding one or more additives to the matrix-forming excipient, the transition temperature properties of the composition may be further desirably affected. For example, the additives may comprise other one or more phospholipids, as described above, and may also comprise added salts that can impact the hydrated and/or the non-hydrated liquid transition temperature of the pharmaceutical composition. For example, one or more polyvalent cations may be added to the pharmaceutical composition to increase the non-hydrated liquid transition temperature. This increase in the non-hydrated liquid transition temperature increases the storage stability of the pharmaceutical composition, reduces the impact of humidity on the pharmaceutical composition, and allows for improved processing of the pharmaceutical composition. Binding of divalent cations to the negatively charged phosphate group of zwitterionic phosphatidylcholines and phosphatidylethanolamines leads to lipids with anionic character. The addition of divalent cations is described in PCT publications WO 01/85136 and WO 01/85137, both of which are incorporated herein by reference in their entireties.

In one version, a polyvalent cation can also be added to the phospholipid matrix forming excipient, such as a divalent cation, for example, one or more of calcium, magnesium, zinc and iron. The polyvalent cation may be present in an amount sufficiently high to increase the liquid transition temperature of the phospholipid composition to a temperature greater than its storage temperature by at least about 20° C., preferably at least about 40° C. The molar ratio of polyvalent cation to phospholipid should be at least about 0.05, preferably from about 0.05 to about 2, and most preferably from about 0.25 to about 1. A molar ratio of polyvalent cation:phospholipid of about 0.5 is particularly preferred, and in one version, the polyvalent cation is calcium. For example, the pharmaceutical composition can have a sufficient amount of calcium chloride to provide a molar ratio of calcium to phospholipid of at least about 0.05, preferably of at least about 0.25, and most preferably of at least about 0.5.

In one particular version, the matrix-forming excipient comprises a lipid component comprising a mixture of phospholipids and a polyvalent cation. For example, the lipid component may comprise a mixture of DMPC and DPPC in an amount sufficient to provide a hydrated liquid transition temperature of just below 37° C., and the pharmaceutical composition may further comprise calcium chloride in a sufficient amount to raise the non-hydrated liquid transition temperature to at least about 80° C., more preferably to at least 90° C. In one version, the lipid component may comprise DMPC in an amount of from about 20% to about 50%, and DPPC in an amount of from about 50% to about 80%, and the calcium may be present in a molar ratio of calcium to phospholipid of about 0.5.

The matrix-forming may comprise an additive to improve the rigidity, production yield, emitted dose and deposition, shelf-life or patient acceptance, of the microparticles. Such optional additives include, but are not limited to coloring agents, taste masking agents, buffers, hygroscopic agents, antioxidants, and chemical stabilizers. Furthermore, various excipients may be incorporated in, or added to, the matrix forming excipient to modify the structure of the microparticles. Such excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids can also be used. The inclusion of both inorganic (e.g. sodium chloride, etc.), organic acids and their salts (e.g. carboxylic acids and their salts such as sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated. Yet other potential additives include particulate compositions that may comprise, or may be coated with, charged species that prolong residence time at the point of contact or enhance penetration through mucosae. For example, anionic charges are known to favor mucoadhesion while cationic charges may be used to associate the formed microparticle with negatively charged biopharmaceutical agents such as genetic material. The charges may be imparted through the association or incorporation of polyanionic or polycationic materials such as polyacrylic acids, polylysine, polylactic acid and chitosan.

Targeting agents that direct the microparticles to cellular targets, such as pulmonary macrophages, can also be added. These agents are particularly useful when the microparticles are administered to treat an infectious disease where a pathogen is taken up by pulmonary macrophages. Such infectious diseases are difficult to treat with conventional systemic treatment with anti-infective pharmaceutical agents. However, by incorporating a targeting agent, the sustained release microparticles are readily absorbed by the pulmonary macrophage, and consequently, more effectively delivered to the site of infection. This method of treatment is particularly effective for the treatment of tuberculosis, bio-warfare agents, such as anthrax, and some types of cancer. The targeting agents may comprises, for example, one or more of phosphatidylserine, hlgG, and muramyl dipeptide, as described in PCT publications WO 99/06855, WO 01/64254, WO 02/09674, and WO 02/87542 and in U.S. Pat. No. 6,630,169, all of which are incorporated herein by reference in their entireties. The targeting process can be more effective if the pharmaceutical agent remains in the lungs for a long period of time. Accordingly, in one version, the composition comprises a targeting agent and sufficient amounts of the matrix-forming excipient to encapsulate at least 70% of a pharmaceutical agent useful to treat an infectious disease where a pathogen is taken up by pulmonary macrophages. Particularly when the composition comprises such a targeting agent, the size of the microparticles is preferably less than 6 μm because larger particles are not readily taken up by pulmonary macrophages.

The composition may also comprise a glass-forming excipient that is capable of stabilizing the pharmaceutical agent or the matrix-forming excipient, for example, during the preparation of solid dosage forms. Preferably, the excipient confers good powder dispersibility properties from a dry powder inhaler. Suitable glass-forming excipients may comprise, for example, at least one of a sugar, polyol, amino acid and homo- or hetero-polymers thereof. For example, the glass-forming excipients can be trileucine, sodium citrate, sodium phosphate, ascorbic acid, polyvinyl pyrrolidone, mannitol, sucrose, trehalose, lactose, proline, and povidone. Further, the composition may also comprise other stabilizing excipients, such as salts of divalent metals, such as zinc, calcium and magnesium.

The microparticles can also include a solubilizing agent to increase the solubility of the pharmaceutical agent in the solution used in the preparation of the microparticles. Suitable solubilizing agents may comprise, for example, at least one of cyclodextrin, polyethylene glycol, polyethylene glycol-polypropylene glycol copolymers, and the afore-mentioned surfactants.

### EXAMPLES

The following examples illustrate the preparation of sustained release microparticles comprising pharmaceutical agents that are suitable for delivery to the pulmonary system, and demonstrate the sustained release provided by the microparticles both in vitro and in vivo.

In these examples, light microscopy, laser diffraction or scanning electron microscopy analysis were used to assess the particle size distribution of the microparticles. For example, scanning electron microscopy analysis was performed using a Philips XL 30 Electronic Scanning Electron Microscope (E-SEM) (FEI Company, Hillsboro, Oreg.). The microparticles were also analyzed via differential scanning calorimetry (DSC) to determine the glass transition temperature of the encapsulated microparticles. The amount of free and encapsulated pharmaceutical agent in the prepared microparticles was determined in vitro. The in vitro release kinetics of the lipid compositions were assessed in a system appropriately engineered to mimic lung delivery conditions. The pharmacokinetics of selected compositions was evaluated in a rabbit or a suitable rat model following intratracheal instillation. The plasma pharmacokinetic data were analyzed to determine mean residence time, while drug bioavailability and cumulative absorption was estimated by deconvolution analysis. The particle structure was characterized by small and wide-angle X-ray diffraction.

### Example 1

### Encapsulation of Salmon Calcitonin

In this example, salmon calcitonin (sCT) was encapsulated in DPI matrix-based sustained release microparticles using a mixture of dipalmitoyl phosphatidylcholine (DPPC) and sugar (lactose). Salmon calcitonin is a 32-amino acid peptide, which typically contains little or no ordered structure in aqueous media. However, in the presence of low dielectric constant solvents, it can adopt an extended alpha helix structure comprising almost 50% of the molecule. Without being limited to any mechanism, structure-activity studies have suggested that its lipid-solubilizing ability is related to its ability to form an amphipathic helix, the later is also formed upon contact with lipids, such as DPPC. Ionic bonding appears to be an important component in the binding of the cationic calcitonin to phospholipids. Such ionic interactions have also been claimed to be important in the formation of the amphipathic helix. At the pH of the solution (7.6) calcitonin is positively charged (pI=10.5), while DPPC is negatively charged, thereby promoting the interaction, which favors the formation of the alpha-helix. However, at the solution conditions the amphipathic helix is also favored by the presence of the organic solvent. Due to the clustering of the hydrophobic residues on one face of the helix, it is possible that some also interact with the lipid with via hydrophobic interactions in the presence of organic solvents. These interactions are further favored by the elevated solution temperature. Moreover, the presence of sCT and lactose induce the formation of a glassy matrix, which is expected to stabilize both the phospholipid and the peptide.

A liquid precursor formulation was prepared from a co-solvent system of ethanol and water having the sCT, phospholipid and lactose combined therein. The non-aqueous phase was prepared by dissolving 1.8 g of DPPC in 510 ml of 99.9% purity ethanol and heating to 45° C. under continuous stirring. The aqueous phase was prepared by dissolving 600 mg of lactose and 600 mg of sCT in 90 ml of de-ionized water and heating to 30° C. under continuous stirring. The two solutions were then combined by slowly adding the aqueous to the ethanol solution, to form a clear solution of final total solids concentration of 0.5% w/v. The volume ratio of the ethanol solution to the water solution was 85:15, and a final pH of the combined solution was 7.6. The final solution composition contained 60% w/w DPPC, 20% w/w sCT and 20% w/w lactose.

The precursor formulation was then spray dried in a Büchi model 191 spray-dryer (Postfach, Switzerland). The precursor formulation was fed to the dryer at 5 mL/min and it was atomized with air at 60 psi. The produced droplets were dried at an inlet temperature of 65° C., yielding an outlet temperature of 49° C. No secondary drying was applied to the collected composition.

The prepared microparticles were also analyzed via differential scanning calorimetry (DSC). The results indicated that the composition consisted of a glass with a glass transition temperature of 43.9° C.; such a transition was absent from the thermogram of the pure phospholipid, indicating that the composition is in the glassy state. The sizes of the microparticles as determined by light microscopy ranged from about 1 to about 5 microns.

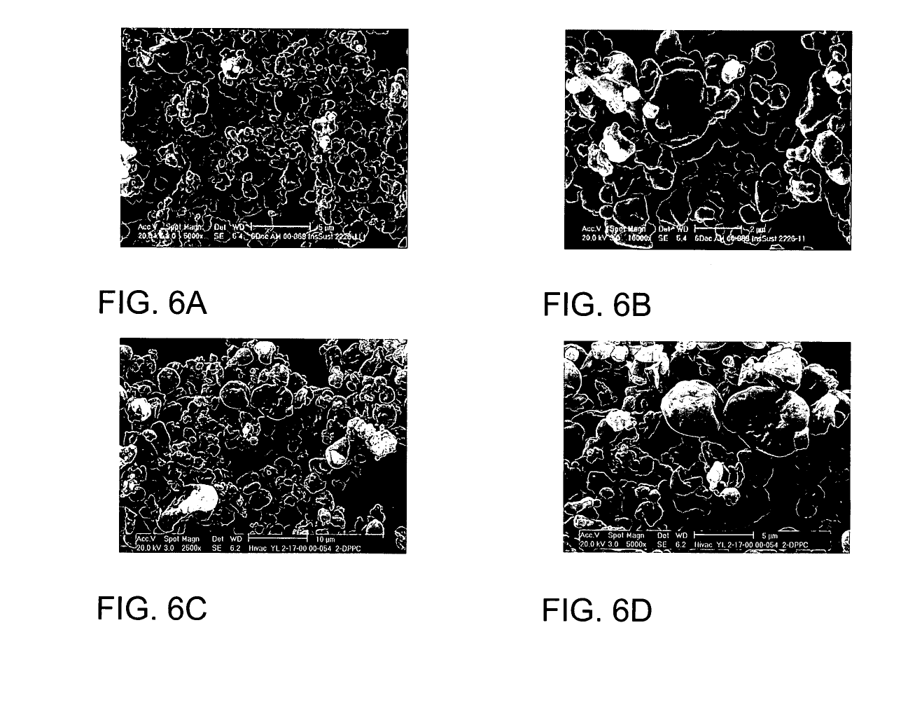

The in vivo dissolution properties of the composition were determined in a rabbit aerosol inhalation model. In this test, the serum sCT concentration was measured for increasing time following intratracheal aerosol administration for sustained release microparticles comprising 20% w/w encapsulated sCT and compared to the concentration of a control sample comprising neat 20% w/w sCT powder. The absorption kinetics, as shown in FIG. 5, indicate that the neat salmon calcitonin is absorbed very rapidly in the lung, reaching its maximum concentration in the blood after 15 minutes following administration, while the blood levels return to baseline after 3 hours. In contrast, the absorption of the encapsulated sCT of the microparticles reaches its maximum blood levels at 4 hours post administration and are sustained to at least 24 hours. These results indicate the substantial alteration of the pharmacokinetic properties of sCT and the retardation of its absorption kinetics. Accordingly, the encapsulated sCT microparticles prepared according to the above method provide improved sustain release results over the unencapsulated sCT, by increasing the duration over which the drug is released to provide a more controlled dose of the sCT drug.

### Examples 2 and 3

### Sustained Release Budesonide in DPPC and DSPC

These examples were conducted to physically encapsulate budesonide between phospholipid bilayers to form a homogeneous solid dispersion of the drug within the lipid matrix. DPPC (dipalmitoyl phosphatidylcholine) and DSPC ( ) were selected as suitable excipients because their transition temperatures are higher than body temperature, Tc of 41° C. and 55° C., respectively. The higher Tc reduces the likelihood of immediate spreading and erosion of inhaled microparticles upon contact with the lung epithelium and its lining fluid, which is at body temperatures.

Preparation

To create homogeneous drug dispersions, budesonide had to be efficiently entrapped within the lipid matrix without phase separation. In Example 2, pulmonary delivery microparticles comprising DPI matrix based compositions of budesonide were prepared using budesonide and DPPC at three different molar ratios. The precursor formulations were prepared from a co-solvent system of ethanol and water with budesonide and DPPC combined therein. The volumetric ratio of ethanol to water in the combined system was about 60:40. The non-aqueous phase was prepared by dissolving (a) 646.75 mg of DPPC and 3.25 mg budesonide (SRB-021), (b) 196 mg DPPC and 4 mg budesonide (SRB-022), and (c) 180 mg DPPC and 20 mg budesonide (SRB-023) in three beakers containing 78, 24 and 24 mL of 99.9% purity ethanol respectively and heating to 45° C. under continuous stirring. The aqueous phases were prepared by heating deionized water to between 30 and 45° C. while stirring. The aqueous phases were slowly added to each ethanol solution in volumes of 52 mL, 16 mL and 16 mL, respectively, to form a clear solution of final total solids concentration of 0.5% w/v. The clear solutions were incubated at 50° C. The incubated precursor formulations were spray dried in a Büchi spray dryer using a nozzle and cyclone. The precursor formulation was fed to the dryer at 5 mL/min and was atomized with clean dry air at 98 psi. The produced droplets were dried at an inlet temperature of 60° C., yielding an outlet temperature of 41° C.

In Example 3, microparticles comprising budesonide entrapped in a DSPC matrix were prepared using a co-solvent system of ethanol and water in a volumetric ratio of ethanol to water of 67:33. The non-aqueous phase was prepared by dissolving 786 mg of DSPC and 16.6 mg of budesonide 107.2 mL of 99.9% purity ethanol each and heating to 65° C. under continuous stirring. The aqueous phases were prepared by heating de-ionized water to 55-60° C. under continuous stirring. The aqueous phase was added slowly in a volume amount of 52.8 mL to the ethanol solution, to form a clear solution having a final total solids concentration of 0.5% w/v. The clear solution was incubated at 65° C. for 1 hour to form the precursor formulation. The composition was then spray dried using the Büchi spray dryer, at a solution feed rate of 5 mL/min and atomized with air at 70 psi. The atomized droplets were dried at an inlet temperature of 60° C. to provide an outlet temperature of 39° C. Secondary drying was not applied to the collected compositions.

Shapes and Sizes of Particle

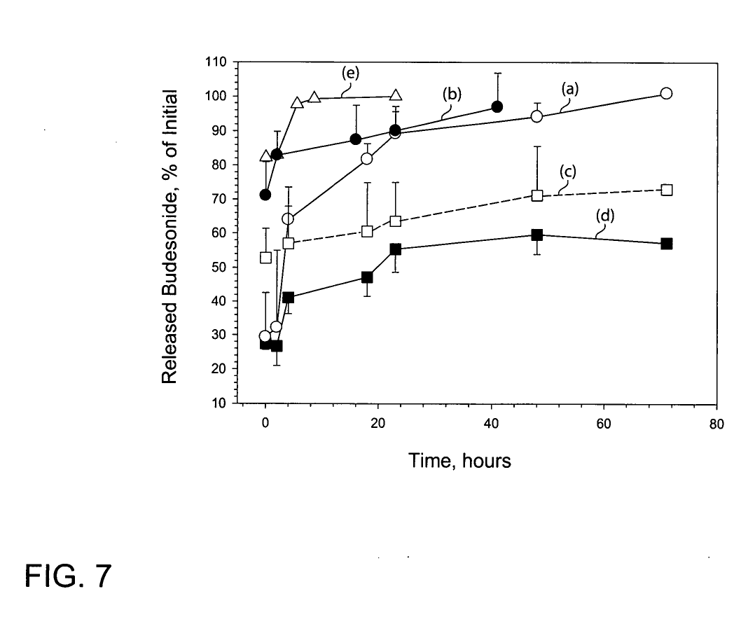

Light microscopy and laser diffraction studies indicated that the microparticles of these examples had particle sizes ranging from about 1 to about 5 micrometers. FIGS. 6A and 6B show scanning electron micrographs of microparticles comprising a DSPC matrix with 2% w/w budesonide, and FIGS. 6C and 6D show microparticles comprising DPPC matrix with 2% w/w budesonide. The SEM micrograph images indicated that microparticles prepared according to both matrix compositions exhibited heterogeneous particle morphologies. The microparticle surfaces appear to be relatively smooth with minor wrinkles, without visible signs of particle fusion, collapse or blowholes in any composition.

The nominal volumetric median diameters (VMD) of the microparticles were determined using laser light scattering on a Malvern Mastersizer X, (Malvern Instruments, Southborough, Mass.). The laser light scattering analysis, as shown in Table I indicates that all compositions exhibited monomodal size distributions with VMDs around 3 to 4 μm.

TABLE I
Physicochemical Characterization of Budesonide Matrix Compositions

| | Nominal | Actual Drug | | Mean |
|---|---|---|---|---|
| | Drug Content | Loading | Drug Burst | Diameter |
| Compositions | (%) | (%) | (%) | (μm) |
| 2% w/w DPPC | 2 | 1.6 | 26.0 ± 1.76 | 3.4 ± 0.1 |
| 5% w/w DPPC | 5 | 2.9 | 34.6 ± 13.3 | 4.0 ± 0.3 |
| 2% w/w DSPC | 2 | 1.2 | 66.9 ± 16.5 | 4.1 ± 0.2 |
| 5% w/w DSPC | 5 | 3.8 | 71.1 ± 10 | — |

Budesonide and Lipid Content

The budesonide content of the DPPC and DSPF microparticles containing budesonide was analyzed via reverse-phase HPLC using a Hewlett Packard (Palo Alto, Calif.) model 1100 HPLC. Budesonide was eluted isocratically through a Symmetry C₁₈ column (Waters, Inc., Milford, Mass.) using a mobile phase consisting of a 45:55 acetonitrile:water mixture at a flow rate of 1 mL/min. Budesonide elution was monitored at 246 nm using a variable wavelength detector. The amount of lipid in the DPPC and DSPF microparticles was determined on a Lichrosorb Diol 5 mm ID 4.6×250 mm column, in normal phase at a flow rate of 0.70 mL/min. The total amount of drug in the compositions was determined by adding 150 μL of 10% w/v Triton X-100 solution to 50 μL of a 1 mg/mL powder suspension in 7 mM Tris-HCl buffer pH 7.4 to solubilize both lipid and drug. The resulting solution was analyzed for both lipid and budesonide contents using the methods described above. The total amount of budesonide in the compositions was expressed as:

$$\% Total \cdot Drug \cdot Loading = \frac{Total Amount of Budesonide}{Budesonide + Lipid} \times 100$$

The DPPC-budesonide microparticles contained 0.5% w/w (SRB-21), 2% w/w (SRB-022) and 10% w/w (SRB-023) budesonide and 99.5, 98 and 90% w/w DSPC, respectively. The DSPC-budesonide contained approximately 2% w/w budesonide and 98% w/w DSP.

In Vitro Dissolution Kinetics & Burst Assessment

The in vitro dissolution kinetics of the matrix compositions were monitored in an automated Vankel (Cary, N.C.) model VK7000 dissolution testing station, equipped with an 8×100-mL USP vessel attachment and an integrated temperature-controlled water bath circulator. The unit was configured as a Type II USP apparatus, using 1.174″ mini Teflon-coated paddles attached to a ¼″ diameter shaft and operated at a speed of 200 rpm. During operation, the desired amount of powder was suspended in 50 mL of the dissolution medium at 37° C., and at appropriate intervals, a 1-mL sample was withdrawn, centrifuged at 12,000 rpm for 10 minutes and the concentration of released budesonide was determined. To account for potential drug losses at selected timepoints, the suspended particles were solubilized by addition of Triton X-100 and the total amount of budesonide was determined.

The in-vitro immediate drug burst was evaluated by incubating 100 □L of 1 mg/mL powder suspension in 7 mM Tris-HCl buffer with 4.9 mL of Survanta in 7 mM Tris-HCl, pH 7.4 buffer for 2 hours at room temperature. The mixture was then centrifuged for 10 minutes at 12-14,000 rpm. The amount of soluble budesonide was determined by HPLC, as described above.

Table I shows the nominal drug content, actual drug loading, drug burst and mean diameter of the microparticles. Regardless of drug loading, the DPPC compositions exhibited lower drug burst (26-35%) compared to their DSPC counterparts (66-71%). This may reflect the increased difficulty of penetration of budesonide in the DSPC bilayers or, alternatively, the increased energy of mixing of the longer phospholipid chains with the drug. Among the DPPC compositions, the one with the lower drug content appeared to have the lower burst, although within experimental error; the same trend was observed with the DSPC compositions. The discrepancy observed between actual drug loading (determined by HPLC analysis) and the calculated nominal is likely due to material losses during the composition preparation and spray drying process.

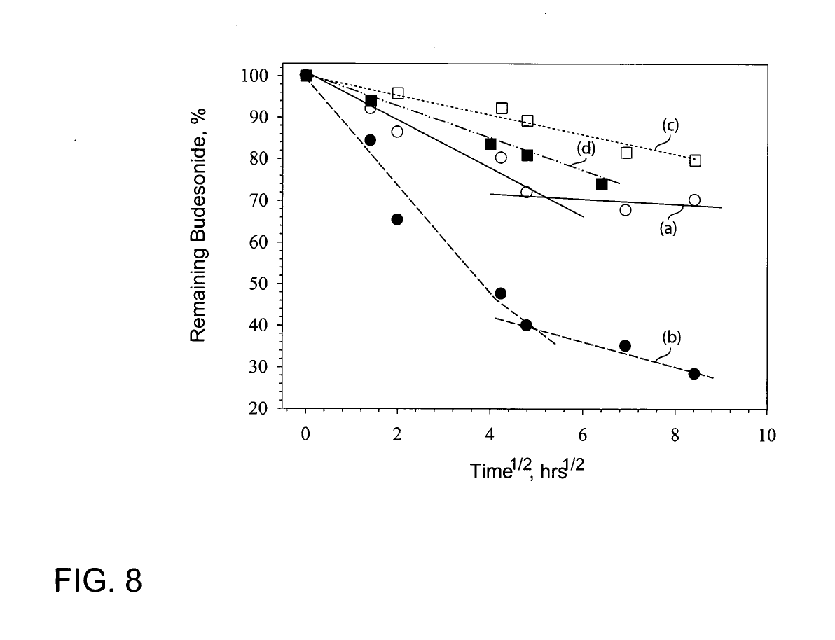

The in vitro dissolution kinetics of the DPPC-budesonide microparticles of Example 2, were monitored using a suitable physiologic buffer medium and with the total budesonide concentration being less than 10% of its solubility in the same media. The in vitro dissolution profiles of budesonide from the lipid matrices over time are shown in FIG. 7. All compositions exhibited a biphasic release profile, with an initial drug burst phase occurring within the first two hours, followed by a second phase which displayed slower release kinetics. It is likely that the majority of budesonide was either free or remained ‘lipid-bound’ on the particle surface. After the initial burst phase, all compositions exhibited a more prolonged dissolution phase, which appeared to follow bi-exponential kinetics. For both phospholipid types, cumulative release was higher for the higher drug loading preparations, which likely reflects their drug entrapment capacity limit. Overall, the phospholipid type did not appear to influence dissolution kinetics from these particles. However, the above data suggests that the concept of budesonide dispersion within lipid matrices is successful in prolonging drug dissolution in vitro, while, outside of the initial burst, the release kinetics appear to be independent of drug loading.

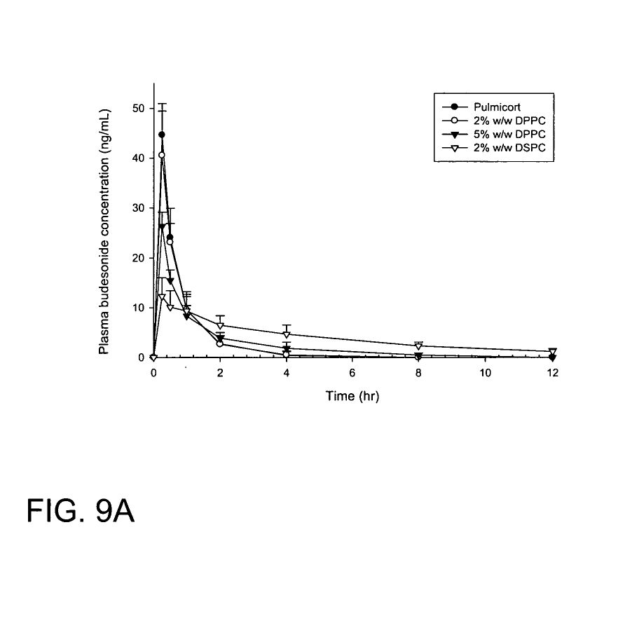

FIG. 8 shows the amount of budesonide released as a percent of the total budesonide for increasing time. The bimodal release pattern was characterized by first correcting for the immediate drug burst at t=0 and then plotting the percent drug remaining as a function of the square root of time (t^{1/2}), to evaluate conformity with the Higuchi diffusion model, described by the equation:
Q=2C₀(Dt/π)^{1/2}
where Q is the amount of released drug, C₀is the initial concentration of budesonide, D is the apparent diffusion coefficient of the drug and t is the elapsed time. Both DSPC compositions exhibited linear t^{1/2} profiles over the entire duration of release (R²=0.973 and 0.9869 for the 2 and 5% w/w compositions, respectively), which supports a primarily diffusion-controlled process. However, two distinct phases can be distinguished for the DPPC compositions. The first one, which extends over the first 24 hours of release, exhibits approximately linear kinetics (R²=0.943 and 0.963 for the 2% and 5% w/w DPPC compositions, respectively) indicating a diffusion-driven process. Thereafter, drug release slows down, suggesting that other parameters govern drug dissolution, such as lipid matrix erosion or, alternatively, the diminished capacity of the dissolution medium as increasing amounts of budesonide become dissolved.

The diffusion-controlled dissolution process is typical of matrix-type delivery systems. The dissolution data was fitted to the Higuchi model, as shown in Table II, and demonstrates a gradually faster drug release from the DPPC compositions at increasing budesonide content.

TABLE II
In Vitro Dissolution Kinetics of Budesonide Matrix Compositions
Fitted to the Higuchi Equation

| | | k | | | CV |
|---|---|---|---|---|---|
| Composition | Model | (hr⁻¹) | n | R² | (%) |
| 2% DPPC | Higuchi | 4.33 ± 0.42 | — | 0.8551 | 9.71 |
| 5% DPPC | Higuchi | 9.86 ± 0.78 | — | 0.8524 | 8.83 |
| 2% DSPC | Higuchi | 2.38 ± 0.11 | — | 0.9764 | 4.59 |
| 5% DSPC | Higuchi | 4.03 ± 0121 | — | 0.9869 | 3.12 |

In Vivo Pharmacokinetic Analysis

The in vivo dissolution properties of the DSPC-budesonide microparticles of Example 3 were determined in a rat suspension instillation model. The concentration of the plasma budesonide was measured for increasing time following single intratracheal instillation in rats for both DSPC encapsulated budesonide and a neat fast acting budesonide control powder (Pulmicort Turbuhaler, Astra). The fast-acting budesonide powder and the three lipid-matrix powder compositions were administered as suspensions in 0.01% w/v Pluronic F-127 in 10 mM sodium phosphate buffer pH 7.4, isotonic with 146 mM sodium chloride. Six male Sprague-Dawley rats (200-450 g) were tested per composition group, at a target dose of 50 μg budesonide/animal delivered through the trachea of the animal. Blood samples (0.40 mL) were collected at pre-determined time intervals to 24 hours and the plasma was separated from the blood Non-compartmental analysis was performed using WinNonLin version 4.1, Pharsight Corp., Palo Alto, Calif. The bioavailability of budesonide was calculated using the amount of drug absorbed at 24 hours estimated by deconvolution analysis. Budesonide was determined in rat plasma using an LC-LC-MS-MS method.

Three lipid-matrix compositions were selected for evaluation in vivo where 2% w/w in DPPC, 5% w/w in DPPC and 2% w/w in DSPC. These were selected to determine the effects of phospholipid chain length and drug concentration in vivo. The selected compositions along with their target and actual label strength and administered dose are given in Table III, in which label strength=(Weight of powder)×(fraction budesonide)/(Volume of Reconstitution)×100.

TABLE III
Characterization of Administered Compositions

| | Budesonide Loading | Label Strength | Actual Dose |
|---|---|---|---|
| Article | (% w/w) | (μg/mL) | (μg) |
| Pulmicort | 100 | 99 | 44.3 (0.005) |
| 2% w/w DPPC | 1.55 | 99.5 | 44.3 (0.18) |
| 5% w/w DPPC | 2.88 | 99.4 | 33.0 (0.9) |
| 2% w/w DSPC | 1.12 | 99.5 | 31.1 (1.0) |

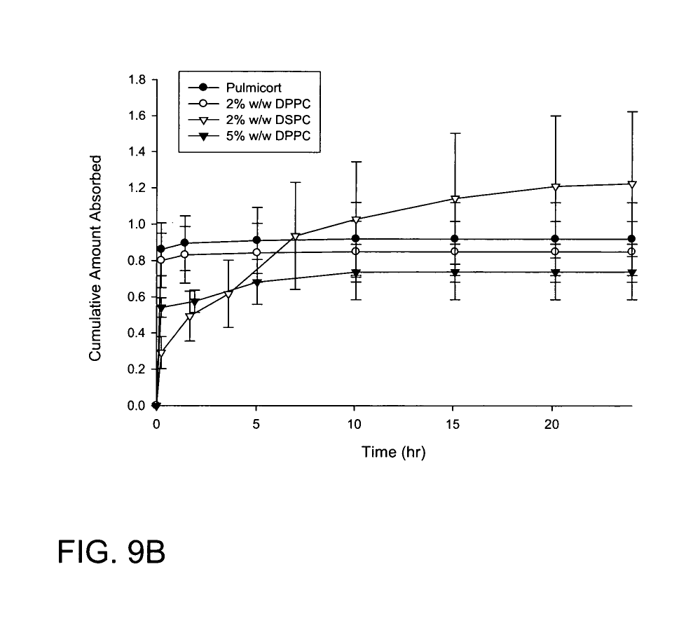

The pharmacokinetic plasma profiles of the microparticles are shown in FIG. 9A, with the parameter values tabulated in Table IV. There were no significant differences between the two DPPC compositions and the fast-releasing commercial Pulmicort powder. Both compositions containing 2% and 5% w/w budesonide in the DPPC-matrix were rapidly absorbed from the lungs, as seen in FIGS. 9A and 9B. However, the 2% w/w budesonide DSPC composition displayed a significantly prolonged MRT compared to Pulmicort, 5.79±1.3 vs. 0.86±0.3 hrs, respectively (P<0.05). The AUC_{inf} for the DSPC composition was significantly higher then the commercial budesonide powder (P<0.05). The mean cumulative amount of budesonide absorbed over time derived from the deconvolution analysis showed that 50% of the bioavailable drug from the Pulmicort powder was absorbed in 0.12±0.01 hrs with 90% being absorbed by 0.71±1.2 hrs (FIG. 9B). The absolute bioavailabilities of budesonide after intratracheal administration of Pulmicort, 2% w/w DPPC and 5% w/w DPPC were 2.1±0.4%, 1.7±0.3% and 2.2±0.5% of the instilled dose, respectively. The bioavailability from the DSPC matrix was significantly higher at 4.2±1.4% of the administered dose (P<0.05). In Table IV, the data was expressed as mean (standard deviation)*p<0.05, 2% budesonide w/w DSPC vs. Pulmicort; and absorption time was for 90% of bioavailability.

TABLE IV
Pharmacokinetic Analysis of Budesonide Compositions

| | | | AUC_{inf} | t_{1/2} | MRT | Absorption |
|---|---|---|---|---|---|---|
| Composition | Cₘₐₓ (ng/mL) | Tₘₐₓ (hr) | (ng/mL * hr) | (hr) | (hr) | Time (hr)^{¶} |
| Pulmicort | 44.6 | 0.25 | 30.4 (10.2) | 0.59 | 0.86 | 0.71 (1.2) |
| | (6.3) | (0.0) | | (0.3) | (0.3) | |
| 2% w/w | 40.5 | 0.25 | 28.9 (8.0) | 0.56 | 0.88 | 3.49 (2.5) |
| DPPC | (8.9) | (0.0) | | (0.3) | (0.3) | |
| 5% w/w | 26.4 | 0.25 | 31.5 (7.7) | 1.96 | 2.29 | 3.40 (2.5) |
| DPPC | (2.8) | (0.0) | | (1.0) | (0.9) | |
| 2% w/w | 12.3 | 0.25 | 55.6 (17.5)* | 4.07 | 5.79 | 12.4 (2.4)* |
| DSPC | (3.7)* | (0.0) | | (0.8)* | (1.3)* | |

Thus, in summary, the DSPC-based composition exhibited prolonged in vivo pharmacokinetics which was 6 to 7 times higher than a releasing commercial powder Pulmicort, with a mean residence time of 5.79±1.3 vs. 0.86±0.3 hrs, respectively. Deconvolution analysis of the PK data revealed an absorption time for 90% of the bioavailable drug of 12.4±2.4 hrs compared to 0.7±1.2 hrs for Pulmicort, which was about 17 times higher.

X-ray Diffraction

To determine the lipid vesicle structure that promotes sustained release, two DSPC compositions (2 and 5% w/w) of Example 3, which exhibited the slowest release kinetics, were characterized via small angle X-ray diffraction (XRD). XRD was 15 performed on the microparticles, using an 18 kWatt Rotating Anode x-ray generator. A bent graphite (002) monochromator was used to focus copper Kα (8 KeV, λ=1.54 Å) radiation on the samples mounted on a 4-circle Huber diffractometer. Vertical slits were used after the sample and before the detector to collect radiation at different scattering angles. The in-plane resolution of the x-ray spectrometer was set by vertical slits to be 0.015 Å-1 (Full-Width-Half-Maximum). Details of the experimental setup are described in “Structure of the Lβ′ Phase in a Hydrated Phosphatidylcholine Multimembrane”, Smith et al., Phys. Rev. Lett. 60, 813 (1988); and “Xray Scattering Studies of Aligned Stacked Surfactant Membranes”, Sirota et al., Science 242, 1406 (1988); both of which are incorporated herein by reference.

All experiments were carried out at room temperature, and each sample was run four separate times for statistics and reproducibility. Small-angle x-ray scattering (SAXS) data (for large length measurements>10 Å) and wide-angle x-ray scattering (WAXS) data (for small length measurements<10 Å) were collected on two compositions. The XRD data, which combines the SAXS and WAXS regions, successfully characterized length scales in the microstructure of the compositions on length scales corresponding to 3 Å<L<125 Å. The samples were each run four times for optimal statistics and reproducibility.

FIG. 3 shows small angle x-ray diffraction (SAXS) patterns of the 2% w/w (solid line) and 5% w/w (dashed line) DSPC-matrix/budesonide compositions. The X-ray intensities are plotted versus q=(4π/λ)sin(2θ/2) where λ=1.54 Å and 2θ is the scattering angle between the incident X-ray beam and the diffracted x-ray beam. The weak peak at a lattice spacing distance of 5.1 Å (indicated with a single arrow) represents locally “melted chains” of the curved regions. The first peak at q₍₀₀₁₎=0.1 (1/Å) corresponds to the (001) peak of the (00L) series; other peaks of the (00L) series at q(004)=0.4 (1/Å), q(005)=0.5 (1/Å), q(006)=0.6 (1/Å), and q(0010)=1.0 (1/Å), are also indicated by solid lines. These peaks arise from the multilamellar structure of the lipids in both DSPC compositions with an inter-lamellar spacing of d=^{2π}/_{q(001)}=62.8 Å.

One surprising aspect of the data is that the (002) and (003) peaks (indicated by dashed lines) are missing. This turns out to be a signature of dry lipid structures, although not the crystalline state.

Wide-angle XRD (WAXD) revealed the highly ordered nature of the lipid lateral packing, indicated by the high angle peaks, illustrated in FIG. 3. A set of three closely spaced peaks is also observed at lattice spacing distances of 4.3 Å, 4.1 Å, and 3.8 Å for both DSPC compositions (for clarity only the peak at 4.1 Å is shown for the 5% composition). These wide-angle peaks are a signature that the phospholipids in both compositions exist in the L_{β′} phase of lipid membranes which is shown schematically in FIG. 1A. The lateral spacing between the lipid chains is from 3.8 Å to 4.3 Å. The XRD data showing sharp peaks at 4.3 Å, 4.1 Å, and 3.8 Å, indicates that the chains are in a highly ordered L_{β′} phase suggests that the pharamaceutical agent is encapsulated in the structural matrix and not inside the hydrophobic region of the chains. The phospholipid bilayer can, for example, have a thickness of from about 25 to about 100 Å. For example, C₁₈ saturated phospholipid chains have a lipid bilayer thickness estimated to be ≈59.9 Å. The total interlayer spacing, as determined in the XRD analysis of the DSPC compositions is 62.8 Å. Thus, the interface gap between the adjacent lipid bilayers is very small, close to ≈2.93 Å (region referred to as d_{w} in FIG. 1A).

The Higuchi-type kinetics displayed by the lipid compositions in vitro suggest a diffusion-controlled release mechanism, which is typical of matrix-type systems. Further, the biphasic release profiles obtained in vitro are in agreement with those reported with liposomal compositions of budesonide, as well as other hydrophobic molecules have shown that release of triamcinolone, a hydrophobic steroid, from liposomes exhibited biphasic release profile which followed Higuchi kinetics. Similarly, drug release from cubic phase gels formed with phospholipids has been proposed to proceed via diffusional exchange of water from the external media of the matrix with water and drug from the interior phases, which follows Higuchi square root of time kinetics.

The rate of drug release from lipid systems is governed by the lipid physicochemical properties, in particular phospholipid chain length, transition temperature and lipid phase. The bilayer curvature and its elasticity in the crystalline lattice structure, whether in the lamellar (L_{□}), reverse hexagonal (H_{II}) or cubic phase (C), will further impact its stability and influence water and drug permeability and matrix degradation. Ultimately, the location of the drug within the lipid matrix will dictate its release. It has been shown that incorporation of steroids within lipid chains, despite their hydrophobic character depends on their lipophilicity, and it has been shown to depend on the molecular geometry of the drug and lipid chain length. In the present examples, budesonide entrapment appeared to increase with decreasing aliphatic chain length, as shown by the reduced immediate burst with the shorter chain DPPC. This presumably occurred due to decreased chain-chain interactions and the increased number of voids in bilayers of DPPC compared to those of DSPC. It has been proposed that due to their structural similarities, location of budesonide and cholesterol, a sterol frequently used to impart stability to the bilayer structure, may be similar. These observations have lead several investigators to suggest, despite the limited impact to its in vitro dissolution kinetics, intercalation of budesonide within the lipid bilayer. Dexamethasone, another hydrophobic corticosteroid, partitions within the DPPC bilayer and that its intercalation is governed by a partition equilibrium between the steroid and liposomes in the aqueous phase. However the X-ray diffraction analysis of the lipid matrix compositions does not support direct interaction of budesonide with the lipid bilayers in this study. The models form a non-liposomal structures that encapsulates the agent between arrangements of lineal or curvelinear bilayers that prolongs release of the agent. The non-liposomal structures can have a linear shape in which both layers of a bilayer are substantially parallel to one another, and can include a layer curled up over itself facing another layer curled in the opposite direction.

The small inter-bilayer spaces available for budesonide intercalation (manifested in the XRD analysis) support findings of low entrapment capacity of budesonide within the lipid structures. Even the compositions prepared at high drug:lipid ratios resulted in relatively low drug entrapment, as observed by the high lipid-to-entrapped drug molar ratios: 30-47:1 and 51:142:1 for DPPC and DSPC, respectively. Increasing budesonide concentration during composition preparation increased drug entrapment in both lipids, as the respective molar ratios increased from 47:1 to 30:1 for DPPC and from 142:1 to 51:1 for DSPC. However, in both cases the composition penalty was the reduced entrapment efficiency, as evidenced by the higher drug burst. The latter may be assigned to the immediate dissolution of un-entrapped budesonide, whether free or bound to the outer lipid surface, along with possible diffusion of drug that may reside within the outer layers of the lipid matrix.

The penalty of reduced drug entrapment in the longer chain DSPC is counterbalanced by the slower drug dissolution rate, owing to its slower ‘diffusion’ from the lipid matrix. In the absence of water from the particle interior, drug diffusivity will largely depend on the degree of disorder of the lipid bilayer, which determines its permeability. This process is expected to be largely dependent on the lipid's transition temperature (T_{c}) in the composition. In turn, the T_{c} of the bilayers in the matrix compositions will depend both on drug interactions but also the deteriorating effect of water ingression during dissolution.

Overall, the XRD data reveals an unexpected, novel multilamellar structure encapsulating the drug with the lipid chains in a well characterized laterally packed conformation. The presence of three rather than two wide-angle chain ordering peaks indicates that the chains are in the L_{βL} phase of multilamellar lipids. In this phase, the lipid chains are tilted with respect to a normal to the lipid bilayer interface at a tilt angle of at least 15°, for example about 30°. Although the chains did not appear to form a 3-dimensional crystal, which is the most ordered and dry phase possible for the chains, they exist in the next most-ordered conformation possible for the lateral packing of the chains in the lipid bilayer. The presence of a broad peak at approximately 5.1 Å may also be explained as resulting from the interaction of the drug molecule with the lipids. It is proposed that budesonide is clustered within small, inter-bilayer interface spaces having a dimension less than 3 Å, such as a thickness of approximately 2.9 Å, which corresponds to a highly dehydrated state, which however does not disturb the strong lateral chain ordering.

The pharmacokinetic assessment of intratracheal instillation of 2% budesonide w/w DSPC-matrix powder composition demonstrated a prolonged retention in the lungs compared to fast-releasing budesonide from the Pulmicort composition. A comparison of the MRT which is a measure of how long an average drug molecule stays in the body shows an increase in the mean MRT from 0.87 hr for Pulmicort to 5.79 hrs with the DSPC composition. The 2% and 5% budesonide DPPC-matrix compositions did not significantly prolong the residence time, with a mean MRT of 0.88 hr and 2.29 hrs, respectively. Deconvolution analysis further supported the finding of a sustained release reservoir in the lung by demonstrating the mean absorption time for 90% of budesonide of 12.36 hrs for the DSPC-matrix compared to 0.71 hrs for Pulmicort.

### Prospective Example 4

In this prospective example, the sustained release microparticles can include an anti-infective active agent, such as ciprofloxacin, various forms of which are described in U.S. Pat. No. 4,670,444 which is incorporated herein by reference in its entirety. Ciprofloxacin is useful in treating infections of in the lungs, such as cystic fibrosis, gram negative infections such as pseudomonas aeruginosa, bronchiectasis, COPD, and chronic bronchitis. Aerosolized ciprofloxacin, when administered to the lungs, has a very short half life. By encapsulating ciprofloxacin as described above, its retention in the lungs is extended and the effectiveness of the active pharmaceutical agent is increased.

In one useful version, the microparticles comprise ciprofloxacin for the purpose of treating a person who has been exposed to inhalation anthrax infection or a person who is in danger of coming into contact with inhalation anthrax. For example, the pharmaceutical composition may be administered to soldiers, to postal workers, or to others who have been or may be exposed to anthrax spores. Endospores of Bacillus anthracis are about 1-2 mm in diameter, optimal for deposition into the deep lung. Endospores are generally phagocytosed by pulmonary macrophages and cleared to mediastinal and peribronchial lymph nodes, where the endospores germinate and release bacilli inside the macrophages. While incubation times are on the order of 10 days, symptoms may occur up to 6 weeks following inhalation, reflecting the ability of endospores to remain in the lungs for extended periods of time. Anthrax bacilli multiply in the lymph nodes, causing hemorrhagic mediastinitis. Eventually the bacteria enter the bloodstream via the thoracic duct, resulting in severe septicemia and often death. Once endospores are cleared to the regional lymph nodes, oral or parenteral treatment with anti-infectives is less efficacious. Local lung delivery allows higher doses of anti-infective, such as the ciprofloxacin to be delivered to the lungs, without correspondingly higher systemic levels, thereby improving the therapeutic index. And most importantly, administration via inhalation is the only way to effectively target the therapeutic to the actual site of the anthrax infection.

By administering the sustained release microparticles with a targeting agent, as described above, the inhalation anthrax can be treated. Ciprofloxacin is currently the anti-infective of choice for treating pulmonary infections of B. anthracis. Ciprofloxacin is a potent and broad-spectrum fluoroquinolone that is especially effective against gram negative pathogens. It is also effective against several pathogens that cause respiratory infections (e.g., Mycobacterium tuberculosis, Mycobacterium avium-M. intracellulare, Hemophilus influenzae, and Pseudomonas aeruginosa).

In one version, high doses of a pharmaceutical composition as described above and comprising ciprofloxacin may be stored in a capsule and administered in a dry powder aerosolization apparatus. Accordingly, the equipment may be easily carried as part of a soldier's military equipment and may be easily stored in a hospital or a postal facility.

### Prospective Example 5

In this example, the microparticles are used to treat mycobacterium, such as tuberculosis. Accordingly, the pharmaceutical agent comprises an anti-tuberculosis agent, such as rifampin and/or isoniazid. Since mycobacterium infections are subject to uptake by pulmonary macrophages, it is preferable for the pharmaceutical composition according to this version to also comprise a targeting agent, as described above.

### Prospective Example 6

Sustained release microparticles can also be used to treat cancer. Accordingly, in this version, the pharmaceutical agent comprises an oncolytic agent, such as one or more of doxorubicin, platinol, paclitaxel, fluorouracil, cytarabine, 9-aminocamptothecin, cyclophosphamide, carboplatin, etoposide, bleomycin, vincristine, vinorelbine, mitomycin-C, and their associated classes and equivalents. Since the uptake of the active agent by pulmonary macrophages may deliver the active agent to the cite of some cancers, it may be preferable in some instances for the pharmaceutical composition according to this version to also comprise a targeting agent, as described above.

### Prospective Example 7

In this example, the sustained release microparticles can comprise a agent which increases the pharmaceutical agent's residence time in the lungs. For example, this agent may comprise one or more asthma agents, such as formoterol and budesonide.

### Prospective Example 8

In another example, the pharmaceutical agent is useful in treating pulmonary Mycobacterium avium-intracellulare (MAI) infections. In this version, a pharmaceutical composition comprising an anti-mycobacterial agent may be administered in a dose of at least 10 mg. The anti-mycobacterial agent is spontaneously encapsulated in the lungs when the pharmaceutical composition is administered to the lungs.

### Prospective Example 9

In another example, the pharmaceutical agent can be useful in treating pulmonary aspergilossis and other fungal infections. In this version, a pharmaceutical agent comprising an anti-fungal agent, such as Amphotericin B, may be administered in a dose of at least 5 mg. The anti-fungal agent is spontaneously encapsulated in the lungs when the pharmaceutical composition is administered to the lungs.

### Prospective Example 10

In another example, the pharmaceutical agent is useful in treating diseases that infect monocytes and macrophages, such as Listeria, Brucella, Leishmania and Mycobacterium avium-intracellulare. Accordingly, in this version, the pharmaceutical anti-infective agent, is one such as amikacin. Since mycobacterium infections are subject to uptake by pulmonary macrophages, it is preferable for the pharmaceutical composition according to this version to also comprise a targeting agent, as described above.

### Prospective Example 11

In another example, the pharmaceutical active agent may be useful in treating Pseudomonas aeruginosa (PA) infections. In this version, the agent comprises an anti-infective agent administered in a dose of at least 5 mg.

In conclusion, it was determined that encapsulation of pharmaceutical agents in microparticles comprising lipid matrices enabled prolonged drug release characterized by an immediate burst and a slower dissolution phase. The microparticles exhibited prolonged in vivo pharmacokinetics which was 6 to 7 times higher than fast releasing commercial powders. Deconvolution analysis revealed an absorption time for 90% of the bioavailable drug of 12.4±2.4 hrs compared to 0.7±1.2 hrs for Pulmicort, which was about 17 times higher. X-ray diffraction analysis of the microparticles revealed the formation of a novel multilamellar structure encapsulating the drug with the lipid chains in a well characterized laterally packed conformation. It is believed that a novel structure comprising pharmaceutical agent clustered within small, inter-bilayer spaces without disturbing the strong lateral chain ordering was formed.

The present invention has been described with reference to certain preferred versions thereof; however, other versions are possible. For example, other the pharmaceutical agents and matrix-forming excipients can be used in other types of applications, as would be apparent to one of ordinary skill. Further, alternative steps equivalent to those described for the matrix-encapsulation forming method can also be used in accordance with the parameters of the described implementation, as would be apparent to one of ordinary skill. Therefore, the spirit and scope of the appended claims should not be limited to the description of the preferred versions contained herein.

What is claimed is:

1. A composition of microparticles for pulmonary delivery, the microparticles comprising:
(a) a pharmaceutical agent; and
(b) a structural matrix comprising a multilamellar structure of lipid bilayers having lipid chains ordered in an L_{βL} phase, the multilamellar structure at least partially encapsulating the pharmaceutical agent at a lipid bilayer interface formed between a plurality of head groups of adjacent lipid bilayers, and capable of providing a sustained release dosage of the pharmaceutical agent.

2. A composition according to claim 1 wherein the multilamellar structure encapsulates the pharmaceutical agent to provide a sustained release dosage of the pharmaceutical agent of at least about 1 mg/hr for at least about 2 hours.

3. A composition according to claim 1 wherein the lipid bilayers comprise phospholipid bilayers.

4. A composition according to claim 3 wherein the phospholipid bilayers are disposed in a lineal arrangement.

5. A composition according to claim 4 wherein the lineal arrangement includes linear sections of phospholipid bilayers and curled sections of phospholipid bilayers.

6. A composition according to claim 5 wherein the multilamellar structure of phospholipid bilayers is absent rotational symmetry.

7. A composition according to claim 3 wherein the multilamellar structure of phospholipid bilayers comprises a non-liposomal structure.

8. A composition according to claim 1 wherein the lipid chains are tilted relative to a normal to the phospholipid bilayer interface at a tilt angle of at least about 15°.

9. A composition according to claim 8 wherein the lipid chains comprise a lateral spacing from one another of from about 3 Å to about 6 Å.

10. A composition according to claim 1 wherein the phospholipid bilayer interface comprises a linear gap between adjacent phospholipid bilayers which are substantially parallel to one another.

11. A composition according to claim 10 wherein each phospholipid bilayer has a thickness of from about 25 to about 100 Å.

12. A composition according to claim 11 wherein a gap at the phospholipid bilayer interface comprises a dimension of less than 3 Å.

13. A composition according to claim 12 wherein the pharmaceutical agent comprises a dimension of less than 3 Å.

14. A composition according to claim 1 wherein the phospholipid bilayer interface comprises an I-shaped gap between phospholipid bilayers that have individual lipid chain layers curling in opposing directions.

15. A composition according to claim 14 wherein the I-shaped gap comprises a thickness of greater than 3 Å.

16. A composition according to claim 15 wherein the pharmaceutical agent comprises a dimension that is larger than 3 Å.

17. A composition according to claim 1 wherein the microparticles exhibit an X-ray diffraction pattern that includes an X-ray diffraction peak corresponding to a lattice spacing distance of 5.1 Å.

18. A composition according to claim 17 wherein the X-ray diffraction pattern further includes X-ray diffraction peaks corresponding to lattice spacing distances of 4.3 Å, 4.1 Å and 3.8 Å.

19. A composition according to claim 1 wherein the multilamellar structure comprises bilayers comprising distearoyl phosphatidylcholine.

20. A composition according to claim 1 wherein the pharmaceutical agent comprises at least one of a steroid, chemotherapeutic agent or anti-infective agent.

21. A composition according to claim 1 wherein the pharmaceutical agent comprises budesonide or salmon calcitonin.

22. A composition according to claim 1 wherein the multilamellar structure encapsulates at least about 0.1% w/w of the pharmaceutical agent.

23. A composition of microparticles for pulmonary delivery, the microparticles comprising:
(a) a pharmaceutical agent; and
(b) a phospholipid structural matrix comprising a multilamellar structure comprising a plurality of phospholipid layers having parallel and tilted lipid chains that are ordered in an L_{βL} phase, the multilamellar structure at least partially encapsulates the pharmaceutical agent in a linear interface gap formed between a first set of head groups of a first phospholipid layer and a second set of head groups of a second phospholipid layer, the first and second phospholipid layers being substantially parallel to one another about the linear interface gap.

24. A composition according to claim 23 wherein the multilamellar structure encapsulates the pharmaceutical agent such that a sustained release of the pharmaceutical agent is provided for at least about 1 hour.

25. A composition according to claim 23 wherein the multilamellar structure encapsulates the pharmaceutical agent to provide a sustained release dosage of the pharmaceutical agent of at least about 1 mg/hr for at least about 2 hours.

26. A composition according to claim 23 wherein the multilamellar structure of phospholipid bilayers comprises non-liposomal structures.

27. A composition according to claim 23 wherein the lipid chains are tilted relative to a normal to the phospholipid bilayer interface at a tilt angle of at least about 15°.

28. A composition according to claim 27 wherein the lipid chains comprise a lateral spacing from one another of from about 3 Å to about 6 Å.

29. A composition according to claim 27 wherein the microparticles exhibit an X-ray diffraction pattern that includes an X-ray diffraction peak corresponding to a lattice spacing distance of 5.1 Å.

30. A composition according to claim 27 wherein the multilamellar structure comprises bilayers comprising distearoyl phosphatidylcholine.

31. A composition according to claim 27 wherein the pharmaceutical agent comprises at least one of a steroid, chemotherapeutic agent or anti-infective agent.

32. A composition according to claim 27 wherein the pharmaceutical agent comprises budesonide or salmon calcitonin.

33. A composition of microparticles for pulmonary delivery, the microparticles comprising:
(a) a pharmaceutical agent; and
(b) a phospholipid structural matrix comprising a multilamellar structure comprising a plurality of phospholipid layers having parallel and tilted lipid chains that are ordered in an L_{βL} phase, the multilamellar structure at least partially encapsulating the pharmaceutical agent in an I-shaped interface gap between a first set of head groups of a first phospholipid layer and a second set of head groups of a second phospholipid layer, the first and second phospholipid layers curling in opposing directions about the I-shaped interface gap.

34. A composition according to claim 33 wherein the multilamellar structure encapsulates the pharmaceutical agent such that a sustained release of the pharmaceutical agent is provided for at least about 1 hour.

35. A composition according to claim 34 wherein the multilamellar structure encapsulates the pharmaceutical agent to provide a sustained release dosage of the pharmaceutical agent of at least about 1 mg/hr for at least about 2 hours.

36. A composition according to claim 33 wherein the multilamellar structure of phospholipid bilayers comprises non-liposomal structures.

37. A composition according to claim 33 wherein the lipid chains are tilted relative to a normal to the phospholipid bilayer interface at a tilt angle of at least about 15°.

38. A composition according to claim 37 wherein the lipid chains comprise a lateral spacing from one another of from about 3 Å to about 6 Å.

39. A composition according to claim 33 wherein the I-shaped gap comprises a thickness of greater than 3 Å.

40. A composition according to claim 39 wherein the pharmaceutical agent comprises a dimension that is larger than 3 Å.

41. A composition according to claim 33 wherein the microparticles exhibit an X-ray diffraction pattern that includes an X-ray diffraction peak corresponding to a lattice spacing distance of 5.1 Å.

42. A composition according to claim 33 wherein the multilamellar structure comprises bilayers comprising distearoyl phosphatidylcholine.

43. A composition according to claim 33 wherein the pharmaceutical agent comprises at least one of a steroid, chemotherapeutic agent or anti-infective agent.

44. A composition according to claim 33 wherein the pharmaceutical agent comprises budesonide or salmon calcitonin.

45. A composition of microparticles for pulmonary delivery, the microparticles comprising:
(a) a pharmaceutical agent; and
(b) a phospholipid structural matrix comprising a multilamellar structure that at least partially encapsulates the pharmaceutical agent to provide a sustained release of the pharmaceutical agent of at least about 1 mg/hr for at least about 2 hours.

46. A composition according to claim 45 wherein the multilamellar structure comprises a plurality of phospholipid layers having parallel and tilted lipid chains that are ordered in an L_{βL} phase.

47. A composition according to claim 45 wherein the multilamellar structure encapsulates the pharmaceutical agent in a linear interface gap formed between a first set of head groups of a first phospholipid layer and a second set of head groups of a second phospholipid layer, the first and second phospholipid layers being substantially parallel to one another about the linear interface gap.

48. A composition according to claim 45 wherein the multilamellar structure encapsulating the pharmaceutical agent in an I-shaped interface gap between a first set of head groups of a first phospholipid layer and a second set of head groups of a second phospholipid layer, the first and second phospholipid layers curling in opposing directions about the an I-shaped interface gap.

49. A composition of microparticles for pulmonary delivery, the microparticles comprising:
(a) a pharmaceutical agent; and
(b) a phospholipid structural matrix comprising a multilamellar structure comprising a plurality of phospholipid layers having parallel and tilted lipid chains that are ordered in an L_{βL} phase, the multilamellar structure at least partially encapsulating the pharmaceutical agent between phospholipid bilayers comprising non-liposomal structures that are disposed in a lineal arrangement which is absent rotational symmetry.

50. A composition according to claim 49 wherein the multilamellar structure encapsulates the pharmaceutical agent such that a sustained release of the pharmaceutical agent is provided for at least about 1 hour.

51. A composition according to claim 50 wherein the multilamellar structure encapsulates the pharmaceutical agent to provide a sustained release dosage of the pharmaceutical agent of at least about 1 mg/hr for at least about 2 hours.

52. A composition according to claim 49 wherein the multilamellar structure of phospholipid bilayers comprises non-liposomal structures.

53. A composition according to claim 49 wherein the lipid chains are tilted relative to a normal to the phospholipid bilayer interface at a tilt angle of at least about 15°.

54. A composition according to claim 53 wherein the lipid chains comprise a lateral spacing from one another of from about 3 Å to about 6 Å.

55. A method of preparing microparticles for pulmonary delivery, the method comprising:
(a) forming a precursor formulation comprising at least one solvent, at least a matrix-forming excipient and a pharmaceutical agent;
(b) heating the precursor formulation to a temperature that is above the liquid-crystalline transition temperature T_{c} of the matrix-forming excipients and below the melting point temperature or denaturation point temperature of the pharmaceutical agent; and
(c) removing the solvent from the precursor formulation to form microparticles suitable for pulmonary delivery, the microparticles comprising a multilamellar structure of the matrix-forming excipient that at least partially encapsulates the pharmaceutical agent.

56. A method according to claim 55 wherein the matrix-forming excipient comprises at least one of a phospholipid, phosphoglycolipid and pegylated phospholipids.

57. A method according to claim 55 wherein the step (b) of heating the precursor formulation is performed prior to the step (c) of removing the solvent to form microparticles.

58. A method according to claim 55 wherein step (c) comprises removing the solvent from the precursor formulation by heating the precursor formulation to a temperature of at least the evaporation point of the solvent.

59. A method according to claim 55 wherein the matrix-forming excipient comprises distearoyl phosphatidylcholine.

60. A method according to claim 55 wherein the pharmaceutical agent comprises at least one of a steroid, chemotherapeutic agent or anti-infective agent.

61. A method according to claim 55 wherein the pharmaceutical agent comprises budesonide or salmon calcitonin.

62. A method according to claim 55 wherein the precursor formulation further comprises a glass-forming excipient comprising at least one of trileucine, sodium citrate, sodium phosphate, ascorbic acid, polyvinyl pyrrolidone, mannitol, sucrose, trehalose, lactose, proline, and povidone.

63. A method according to claim 55 wherein the precursor formulation comprises an active-agent solubilizing excipient comprising at least one of cyclodextrin, polyethylene glycol, polyethylene glycol-polypropylene glycol copolymers, and surfactants.

64. A method according to claim 55 wherein the solution comprises a first solvent and a second solvent, the second solvent being less polar than the first solvent, and wherein the matrix forming excipient is more soluble in the first solvent than the pharmaceutical agent.

65. A method according to claim 55 wherein the first solvent comprises at least one of an alcohol, ketone, chlorinated solvent, ether and a fluorocarbon.

66. A method according to claim 55 wherein the second solvent comprises water.

67. A method according to claim 55 comprising combining a volumetric ratio of the first solvent to the second solvent of from about 99.9:0.1 to about 1:100.

68. A method according to claim 55 wherein the volumetric ratio is from about 70:30 to about 30:70.

69. A method according to claim 55 further comprising heating at least one of the first and second solvents to a temperature above the liquid-crystalline transition temperature T_{c} before combining the first and second solvents to form the solution.

70. A method according to claim 67 wherein (b) comprises maintaining the temperature for at least about 90 minutes.

71. A method according to claim 55 wherein (c) comprises spray drying the solution to form the particles comprising the pharmaceutical agent and the matrix-forming excipient.

72. A composition of microparticles for pulmonary delivery, the microparticles formed according to the method of claim 55.
